SEQUENCE LISTING (57 sequences)

SEQ ID NO: 1 - protein, 5 aa, Synthetic construct
GYAMS

SEQ ID NO: 2 - protein, 17 aa, Synthetic construct
SISGSGGSTYYADSVKG

SEQ ID NO: 3 - protein, 19 aa, Synthetic construct
PPGRQEYYGSSIYYFPLGN

SEQ ID NO: 4 - protein, 11 aa, Synthetic construct
GGNNIGSKSVH

SEQ ID NO: 5 - protein, 7 aa, Synthetic construct
DDSDRPS

SEQ ID NO: 6 - protein, 11 aa, Synthetic construct
QVWDSSSDLVV

SEQ ID NO: 7 - protein, 128 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSS

SEQ ID NO: 8 - protein, 108 aa, Synthetic construct
QSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 9 - protein, 18 aa, Synthetic construct
ASTKGPKLEEGEFSEARV

SEQ ID NO: 10 - protein, 254 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 11 - protein, 5 aa, Synthetic construct
TYAMN

SEQ ID NO: 12 - protein, 19 aa, Synthetic construct
RIRSKYNNYATYYADSVKD

SEQ ID NO: 13 - protein, 14 aa, Synthetic construct
HGNFGNSYVSWFAY

SEQ ID NO: 14 - protein, 14 aa, Synthetic construct
RSSTGAVTTSNYAN

SEQ ID NO: 15 - protein, 7 aa, Synthetic construct
GTNKRAP

SEQ ID NO: 16 - protein, 9 aa, Synthetic construct
ALWYSNLWV

SEQ ID NO: 17 - protein, 123 aa, Synthetic construct
EVKLLESGGLVQPKGSLKLSCAASGFTFNTYAMNWVRQAPGKGLEWVARIRSKYNNYATYYADSVKDRFTISRDDSQSILYLQMNNLKTEDTAMYYCRHGNFGNSYVSWFAYWGQGTLVTVSA

SEQ ID NO: 18 - protein, 109 aa, Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 19 - protein, 125 aa, Synthetic construct
EVQLVESGGGLVQPGGSLRLSCAASGFTFSTYAMNWVRQAPGKGLEWVGRIRSKYNNYATYYAASVKDRFTISRDDSKNSLYLQMNSLKTEDTAVYYCARHGNFGNSYVSWFAYWGQGTLVTVSS

SEQ ID NO: 20 - protein, 125 aa, Synthetic construct
EVQLVESGGGLVQPGGSLRLSCAASGFTFNTYAMNWVRQAPGKGLEWVGRIRSKYNNYATYYAASVKDRFTISRDDSKSSLYLQMNSLKTEDTAVYYCARHGNFGNSYVSWFAYWGQGTLVTVSS

SEQ ID NO: 21 - protein, 125 aa, Synthetic construct
EVQLVESGGGLVQPGGSLRLSCAASGFTFNTYAMNWVRQAPGKGLEWVARIRSKYNNYATYYAASVKDRFTISRDDSQNSLYLQMNSLKTEDTAVYYCVRHGNFGNSYVSWFAYWGQGTLVTVSS

SEQ ID NO: 22 - protein, 125 aa, Synthetic construct
EVKLVESGGGLVQPGGSLRLSCAASGFTFNTYAMNWVRQAPGKGLEWVARIRSKYNNYATYYAASVKDRFTISRDDSQSSLYLQMNSLKTEDTAVYYCVRHGNFGNSYVSWFAYWGQGTLVTVSS

SEQ ID NO: 23 - protein, 125 aa, Synthetic construct
EVQLLESGGGLVQPGGSLRLSCAASGFTFSTYAMNWVRQAPGKGLEWVSRIRSKYNNYATYYAASVKDRFTISRDDSKNTLYLQMNSLRAEDTAVYYCARHGNFGNSYVSWFAYWGQGTLVTVSS

SEQ ID NO: 24 - protein, 125 aa, Synthetic construct
EVKLLESGGGLVQPGGSLRLSCAASGFTFNTYAMNWVRQAPGKGLEWVARIRSKYNNYATYYAASVKDRFTISRDDSKNTLYLQMNSLRAEDTAVYYCARHGNFGNSYVSWFAYWGQGTLVTVSS

SEQ ID NO: 25 - protein, 109 aa, Synthetic construct
QTVVTQEPSLTVSPGGTVTLTCRSSTGAVTTSNYANWVQQKPGQAPRALIGGTNKRAPWTPARFSGSLLGDKAALTLSGVQPEDEAEYYCALWYSNLWVFGGGTKLTVL

SEQ ID NO: 26 - protein, 109 aa, Synthetic construct
QTVVTQEPSLTVSPGGTVTLTCRSSTGAVTTSNYANWVQQKPGQAPRGLIGGTNKRAPGTPARFSGSLLGDKAALTLSGVQPEDEAEYYCALWYSNLWVFGGGTKLTVL

SEQ ID NO: 27 - protein, 109 aa, Synthetic construct
QAVVTQEPSLTVSPGGTVTLTCRSSTGAVTTSNYANWVQQKPGQAPRGLIGGTNKRAPGTPARFSGSLLGDKAALTLSGVQPEDEAEYFCALWYSNLWVFGGGTKLTVL

SEQ ID NO: 28 - protein, 109 aa, Synthetic construct
QTVVTQEPSFSVSPGGTVTLTCRSSTGAVTTSNYANWYQQTPGQAPRTLIGGTNKRAPGVPDRFSGSILGDKAALTITGAQADDESDYYCALWYSNLWVFGGGTKLTVL

SEQ ID NO: 29 - protein, 109 aa, Synthetic construct
QTVVTQEPSFSVSPGGTVTLTCRSSTGAVTTSNYANWVQQTPGQAPRGLIGGTNKRAPGVPDRFSGSILGDKAALTITGAQADDESDYYCALWYSNLWVFGGGTKLTVL

SEQ ID NO: 30 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Synthetic construct [sequence omitted]

SEQ ID NO: 33 - protein, 5 aa, Synthetic construct
GGGGS

SEQ ID NO: 34 - protein, 5 aa, Synthetic construct
GGGSG

SEQ ID NO: 35 - protein, 5 aa, Synthetic construct
GSGGG

SEQ ID NO: 36 - protein, 7 aa, Synthetic construct
GSGGGGP

SEQ ID NO: 37 - protein, 4 aa, Synthetic construct
GSEP

SEQ ID NO: 38 - protein, 7 aa, Synthetic construct
GGEGGGP

SEQ ID NO: 39 - protein, 12 aa, Synthetic construct
GGEGGGSEGGGS

SEQ ID NO: 40 - protein, 8 aa, Synthetic construct
GGGSGGGG

SEQ ID NO: 41 - protein, 729 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVLGGGGSGGGGSGGGGSGGGGSEVQLVESGGGLVQPGGSLRLSCAASGFTFSTYAMNWVRQAPGKGLEWVGRIRSKYNNYATYYAASVKDRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 729 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVLGGGGSGGGGSGGGGSGGGGSEVQLVESGGGLVQPGGSLRLSCAASGFTFNTYAMNWVRQAPGKGLEWVGRIRSKYNNYATYYAASVKDRFTISRDDSKSSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 729 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVLGGGGSGGGGSGGGGSGGGGSEVQLVESGGGLVQPGGSLRLSCAASGFTFNTYAMNWVRQAPGKGLEWVARIRSKYNNYATYYAASVKDRFTISRDDSQNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 729 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVLGGGGSGGGGSGGGGSGGGGSEVKLVESGGGLVQPGGSLRLSCAASGFTFNTYAMNWVRQAPGKGLEWVARIRSKYNNYATYYAASVKDRFTISRDDSQSSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 729 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVLGGGGSGGGGSGGGGSGGGGSEVQLLESGGGLVQPGGSLRLSCAASGFTFSTYAMNWVRQAPGKGLEWVSRIRSKYNNYATYYAASVKDRFTISRDDSKNTLYLQMNSLRAEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 729 aa, Synthetic construct
EVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVLGGGGSGGGGSGGGGSGGGGSEVKLLESGGGLVQPGGSLRLSCAASGFTFNTYAMNWVRQAPGKGLEWVARIRSKYNNYATYYAASVKDRFTISRDDSKNTLYLQMNSLRAEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 729 aa, Synthetic construct
EVQLVESGGGLVQPGGSLRLSCAASGFTFSTYAMNWVRQAPGKGLEWVGRIRSKYNNYATYYAASVKDRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 48 - protein, 729 aa, Synthetic construct
EVQLVESGGGLVQPGGSLRLSCAASGFTFNTYAMNWVRQAPGKGLEWVGRIRSKYNNYATYYAASVKDRFTISRDDSKSSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 49 - protein, 729 aa, Synthetic construct
EVQLVESGGGLVQPGGSLRLSCAASGFTFNTYAMNWVRQAPGKGLEWVARIRSKYNNYATYYAASVKDRFTISRDDSQNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 50 - protein, 729 aa, Synthetic construct
EVKLVESGGGLVQPGGSLRLSCAASGFTFNTYAMNWVRQAPGKGLEWVARIRSKYNNYATYYAASVKDRFTISRDDSQSSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 51 - protein, 729 aa, Synthetic construct
EVQLLESGGGLVQPGGSLRLSCAASGFTFSTYAMNWVRQAPGKGLEWVSRIRSKYNNYATYYAASVKDRFTISRDDSKNTLYLQMNSLRAEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 52 - protein, 729 aa, Synthetic construct
EVKLLESGGGLVQPGGSLRLSCAASGFTFNTYAMNWVRQAPGKGLEWVARIRSKYNNYATYYAASVKDRFTISRDDSKNTLYLQMNSLRAEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLLQPGGSLRLSCAASGFTFSGYAMSWVRQAPGKGLEWVSSISGSGGSTYYADSVKGRFTISRDNSKNTLYLQMNSLRAEDTALYYCAKPPGRQEYYGSSIYYFPLGNWGQGTLVTVSSASTKGPKLEEGEFSEARVQSALTQPASVSVAPGQTARITCGGNNIGSKSVHWYQQKPGQAPVLVVYDDSDRPSGIPERFSGSNSGNTATLTISRVEAGDEADYYCQVWDSSSDLVVFGGGTKLTVL

SEQ ID NO: 53 - protein, 216 aa, Synthetic construct
QTVVTQEPSLTVSPGGTVTLTCRSSTGAVTTSNYANWVQQKPGQAPRALIGGTNKRAPWTPARFSGSLLGDKAALTLSGVQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 216 aa, Synthetic construct
QTVVTQEPSLTVSPGGTVTLTCRSSTGAVTTSNYANWVQQKPGQAPRGLIGGTNKRAPGTPARFSGSLLGDKAALTLSGVQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 216 aa, Synthetic construct
QAVVTQEPSLTVSPGGTVTLTCRSSTGAVTTSNYANWVQQKPGQAPRGLIGGTNKRAPGTPARFSGSLLGDKAALTLSGVQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 216 aa, Synthetic construct
QTVVTQEPSFSVSPGGTVTLTCRSSTGAVTTSNYANWYQQTPGQAPRTLIGGTNKRAPGVPDRFSGSILGDKAALTITGAQADDESDYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 216 aa, Synthetic construct
QTVVTQEPSFSVSPGGTVTLTCRSSTGAVTTSNYANWVQQTPGQAPRGLIGGTNKRAPGVPDRFSGSILGDKAALTITGAQADDESDYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0382352 A1
(43) Pub. Date: Dec. 18, 2025

(54) BISPECIFIC ANTIBODIES FOR HBV SURFACE ANTIGEN (HBSAG) AND CD3 AND USES THEREOF

(71) Applicant: SCG CELL THERAPY PTE. LTD, Singapore (SG)

(72) Inventors: Pin XIE, Shanghai (CN); Ke ZHANG, Shanghai (CN)

(21) Appl. No.: 18/879,271

(22) PCT Filed: Jun. 26, 2023
(86) PCT No.: PCT/SG2023/050449

§ 371 Date: Dec. 27, 2024

(30) Foreign Application Priority Data
Jun. 27, 2022 (CN) 202210745544.5

(51) Int. Cl. C07K 16/08; A61K 39/00; A61P 31/20; C07K 16/28

(52) U.S. Cl. CPC C07K 16/082; A61P 31/20; C07K 16/2809; A61K 2039/505; C07K 2317/31; C07K 2317/565; C07K 2317/622; C07K 2317/76; C07K 2317/92

(57) ABSTRACT

Provided are bispecific antibodies that may specifically target both HBV surface antigen (HBsAg) and CD3 protein and pharmaceutical compositions comprising the same. Particularly, provided are nucleic acid molecules encoding the bispecific antibodies or fragments thereof and vectors including the nucleic acid molecules. Also provided uses of the bispecific antibodies or fragments thereof and the nucleic acid molecules and the vectors in preparation of medicines for preventing or treating HBV infection and other related diseases.

### FIELD OF THE INVENTION

The present invention relates to bispecific homodimeric antibodies and uses thereon in the treatment of Hepatitis B virus infection and associated hepatocellular carcinoma.

### BACKGROUND OF THE INVENTION

Globally more than 400 million people have been infected by hepatitis B virus, which represents 3.5% of the total population, and the highest prevalence is about 6% in Western Pacific region and African region according to WHO's data. Acute HBV infection may develop into chronic hepatitis B virus (CHB) (90% of newborn and 5% of adult patients, respectively) with poor prognosis, consequently resulting in cirrhosis, liver failure and hepatocellular carcinoma (HCC) with 6000,000 deaths each year.

Current clinical management of CHB patients involves antiviral drugs, like tenofovir and entecavir, to suppress viral replication but not to eliminate HBV covalently closed circular DNA (cccDNA) carrying hepatocytes, which may recall HBV infection once medicine intake stopped. It has been reported that clearance of HBV infection is associated with sustained viral control by effector T cells, while progression of chronic infection is believed to be due to limited virus specific T cell responses. Therefore, there is an urgent need to develop a novel immune therapy approach for CHB to restore HBV specific T cell mediated response against virus infected liver cells.

T cell redirecting bispecific antibody refers to a molecule comprising more than two binding domains, wherein a first domain specifically binds to a cell surface antigen (such as tumor associated/pathogenic specific) on a target cell/tissue and wherein a second domain of the molecule specifically binds to a T cell antigen (e.g., CD3). This dual target binding molecule can redirect T cells to the target cell/tissue, leading to the elimination of the target cells.

CD3 refers to a human antigen which is expressed on T cells as part of TCR/CD3 complex of T-lymphocytes comprising either a TCR alpha/beta or TCR gamma/delta heterodimer co-expressed at the cell surface with the invariant subunits of CD3 labeled gamma, delta, epsilon and zeta. Human CD3ε is described under UniProtP07766 (CD3E_HUMAN). An anti CD3ε antibody described in the state of the art is SP34 (Yang S J, The Journal of Immunology (1986) 137; 1097-1100). SP34 is available from Pharmigen. A further anti CD3 antibody described in the state of the art is UCHT-1 (seen in WO2000041474). A further anti CD3 antibody described in the state of the art is BC-3 (Fred Hutchison Cancer Research Institute; used in Phase I/II trials of GvHD, Anasetti et al. Transplantation 54:844 (1992)). SP34 differs from UCHT-1 and BC3 in that SP34 recognizes an epitope present on solely the & chain of CD3 (seen in Salmeron et al., (1991)) whereas UCHT-1 and BC-3 recognize an epitope contributed by both the ε and δ chains. The sequence of an antibody with the same sequences as of antibody SP34 is mentioned in WO2008119565, WO2008119566, WO2008119567. WO2010037836, WO2010037837, and WO2010037838. A sequence which is 96% identical to VH of antibody SP34 is mentioned in U.S. Pat. No. 8,236,308 (WO2007042261). However, there are the problem of big adverse reaction and poor draggability for many existing CD3 antibodies including SP34.

HBsAg refers to the envelope antigen of hepatitis B virus displaying in the surface of viral infected cells. HBV S/L/M surface proteins are the small, medium, and large surface proteins, which are transcribed and translated from one reading frame and differ from each other by N-terminal part. Accordingly, the large surface antigen comprises a part which is neither present in the medium nor in the small surface antigen, and the medium surface antigen comprises a part which being comprised in the large antigen but not comprised in the small antigen. The small antigen consists of a sequence, which is comprised in the C-terminal part of both the medium and the large antigen. It's presumed that this occurs although the virus is released into intracellular vesicles because numbers of HBV surface proteins (HBsAg) remain integrated into the intracellular membrane of the endoplasmic reticulum. During vesicle transport processes said intracellular membrane may fuse with the cellular membrane, the consequence being that HBV surface proteins are displayed on the surface of the infected cells. Therefore, HBsAg provides an ideal target for virus aimed immunotherapeutic intervention to treat CHB.

### SUMMARY OF THE INVENTION

The present application provides a structural form suitable for anti-HBsAg/CD3 bispecific antibody, wherein one antibody is in the form of IgG, the other in the form of scFv or VHH nanoantibody form. Preferably, the CD3 antibody is in the IgG1 structural form, the HBsAg antibody is in the scFv form and fused at the N-terminal or C-terminal of the CD3 antibody heavy chain. On the other hand, the present application further provides a CD3 antibody or an antigen-binding fragment thereof significantly reduced affinity to CD3 and a bispecific or a multispecific antigen-binding molecule comprising a CD3 antibody or an antigen-binding fragment thereof.

The first aspect of the present invention provides for a T cell redirecting bispecific homomeric molecule anti-HBsAg×anti-CD3, wherein the bispecific antibody comprising:
(a) a first polypeptide chain comprising a heavy chain of the anti-CD3 antibody (anti-CD3 VH-CH1-Fc) fused with a single chain fragment (scFv) binding to hepatitis B virus surface antigen (HBsAg), wherein the HBsAg comprises small surface antigen, hepatitis B virus medium surface antigen, or hepatitis B virus large surface antigen.
(b) a second polypeptide chain comprising a light chain of the anti-human CD3 antibody (anti CD3 VL-CL).

In some embodiments, the HBsAg scFv, is fused to the N-terminus or C-terminus of the heavy chain of the anti-CD3 antibody; preferably, fused to the C-terminus of the heavy chain of the anti-CD3 antibody. In some embodiments, the HBsAg scFv is fused to the N-terminus or C-terminus of the heavy chain of the anti-CD3 antibody through a glycine-serine linker. In some embodiments, the glycine-serine linker is selected from (GGGGS)n of SEQ ID NO: 33, (GGGSG)n of SEQ ID NO: 34, (GSGGG)n of SEQ ID NO: 35, (GSGGGP)n of SEQ ID NO: 36, (GSEPS)n of SEQ ID NO: 37, (GGEGGGP)n of SEQ ID NO: 38, (GGEGGGSEGGGS)n of SEQ ID NO: 39, (GGGSGGGG)n of SEQ ID NO: 40 or a combination thereof, wherein the n=1, 2, 3, 4, 5, 6, 7, 8, 9 or 10.

In some embodiments, the HBsAg scFv comprises the HCDR1-3, the LCDR1-3 as shown in SEQ ID NO: 1 to SEQ ID NO: 6, respectively.

In some embodiments, the CD3 antibody comprises the HCDR1-3, the LCDR1-3 as shown in SEQ ID NO: 11 to SEQ ID NO: 16, respectively.

In some embodiments, the HBsAg scFv comprises a VH binding domain and a VL binding domain; wherein the VH comprises the amino acid sequence that is at least 90% identical to SEQ ID NO: 7; the VL comprises the amino acid sequence that is at least 90% identical to SEQ ID NO: 8.

In some embodiments, the VH domain and VL domain are linked by a “YOL” linker; preferably, the “YOL” linker comprises the amino acid sequence of SEQ ID NO: 9.

In some embodiments, the HBsAg scFv comprises the amino acid sequence that is at least 90% identical to SEQ ID NO: 10.

In some embodiments, the CD3 antibody fraction of the HBsAg/CD3 double specific antibody is a humanized antibody with a significantly reduced affinity for CD3. In some embodiments, the humanized anti-human CD3 antibody includes a VH domain and a VL domain; wherein the VH comprises the amino acid sequence that is at least 90% identical to SEQ ID NO: 19, SEQ ID NO: 20, SEQ ID NO: 21, SEQ ID NO: 22, SEQ ID NO: 23, or SEQ ID NO: 24; In some embodiments, the VL comprises the amino acid sequence that is at least 90% identical to SEQ ID NO: 25, SEQ ID NO: 26, SEQ ID NO: 27, SEQ ID NO: 28, or SEQ ID NO: 29.

In some preferred embodiments, the amino acid sequence of the VL domain of the CD3 antibody as shown in SEQ ID NO: 25. In some other preferred embodiments, the VH domain amino acid sequences of the CD3 antibody comprises SEQ ID NO: 19, SEQ ID NO: 20, SEQ ID NO: 21, SEQ ID NO: 22, SEQ ID NO: 23 or SEQ ID NO: 24, and the amino acid sequence of the VL domain of the CD3 antibody as shown in SEQ ID NO: 25 or having at least 90% sequence identity with it.

In some embodiments, the bispecific antibody comprises heavy chain constant region and its conventional variants selected from the IgG1 subtype, IgG2 subtype, IgG3 subtype, or IgG4 subtype, and light chain constant region and its conventional variants selected from the κ or λ subtype. Preferably, the constant region belongs to IgG1-kappa isotype. In some embodiments, the bispecific antibody further comprises alanine at position 234 and alanine at position at 235 of heavy chain, wherein residue numbering is according to the EU Index.

In some embodiments, the bispecific antibody comprises a first polypeptide chain and a second polypeptide chain, wherein: (a). the first polypeptide chain comprises, in the N-terminal to C-terminal direction: i. A domain I, comprising three subdomains, wherein subdomain IA comprised a variable heavy region (VH) binds to an epitope of hepatitis B virus small surface antigen, hepatitis B virus medium surface antigen, or hepatitis B virus large surface antigen, set as forth in SEQ ID NO: 7; wherein subdomain IB comprises a “YOL” linker sequence set as forth in SEQ ID NO: 9; wherein subdomain IC comprised a variable light region (VL) binds to an epitope of hepatitis B virus small surface antigen, hepatitis B virus medium surface antigen, or hepatitis B virus large surface antigen, set as forth in SEQ ID NO: 8; ii. A domain II comprised a glycine-serine linker sequence set as forth in any one of SEQ ID NO: 33-40, wherein the copy number n=“1-10”; iii. A domain III, wherein a variable heavy region (VH) binds to an epitope of human CD3 sequence set as forth in any one of SEQ ID NO: 19-24; iv. A domain IV, wherein a constant region of heavy chain sequence set as forth in SEQ ID NO: 30, or SEQ ID NO: 31. (b) the second polypeptide chain comprises, in the N-terminal to C-terminal direction: i. A domain I, wherein a variable light region (VL) binds to an epitope of human CD3 sequence set as forth in any one of SEQ ID NO: 25-29; ii. a domain II, wherein a constant region of light chain sequence set as forth in SEQ ID NO: 32.

In some embodiments, the bispecific antibody comprises a first polypeptide chain and a second polypeptide chain, wherein: (a). the first polypeptide chain comprises, in the N-terminal to C-terminal direction: i. A domain I, wherein a variable heavy region (VH) binds to an epitope of human CD3 sequence set as forth in any one of SEQ ID NO: 19-24; ii. A domain II, wherein a constant region of heavy chain sequence set as forth in SEQ ID NO: 30, or SEQ ID NO: 31; iii. A domain III, comprised a glycine-serine linker sequence set as forth in any one of SEQ ID NO: 33-40, wherein the copy number n=“1-10”; iv. A domain IV, comprising three subdomains, wherein subdomain IVA comprised a variable heavy region (VH) binds to an epitope of hepatitis B virus small surface antigen, or hepatitis B virus medium surface antigen, or hepatitis B virus large surface antigen, set as forth in SEQ ID NO: 7; wherein subdomain IVB comprised a “YOL” linker sequence set as forth in SEQ ID NO: 9; wherein subdomain IVC comprised a variable light region (VL) binds to an epitope of hepatitis B virus small surface antigen, or hepatitis B virus medium surface antigen, or hepatitis B virus large surface antigen, set as forth in SEQ ID NO: 8. (b). A second polypeptide chain comprises, in the N-terminal to C-terminal direction: i. A domain I, wherein a variable light region (VL) binds to an epitope of human CD3 sequence set as forth in any one of SEQ ID NO: 25-29; ii. a domain II, wherein a constant region of light chain sequence set as forth in SEQ ID NO: 32.

In some embodiments, the bispecific antibody comprises a first polypeptide chain and a second polypeptide chain, wherein: (a). the first polypeptide chain comprises, in the N-terminal to C-terminal direction: i. A domain I, comprising single chain Fv region (scFv) set as forth in SEQ ID NO: 10, wherein the scFv fragment binds to an epitope of hepatitis B virus small surface antigen, or hepatitis B virus medium surface antigen, or hepatitis B virus large surface antigen; ii. A domain II comprised a glycine-serine linker sequence set as forth in any one of SEQ ID NO: 33-40, wherein the copy number n=“1-10”; iii. A domain III, wherein a variable heavy region (VH) binds to an epitope of human CD3 sequence set as forth in any one of SEQ ID NO: 19-24; iv. A domain IV, wherein a constant region of heavy chain sequence set as forth in SEQ ID NO: 30, or SEQ ID NO: 31. (b). the second polypeptide chain comprises, in the N-terminal to C-terminal direction: i. A domain I, wherein a variable light region (VL) binds to an epitope of human CD3ε sequence set as forth in any one of SEQ ID NO: 25-29; ii. a domain II, wherein a constant region of light chain sequence set as forth in SEQ ID NO: 32.

In some embodiments, the bispecific antibody comprises a first polypeptide chain and a second polypeptide chain, wherein: (a). the first polypeptide chain comprises, in the N-terminal to C-terminal direction: i. A domain I, wherein a variable heavy region (VH) binds to an epitope of human CD3 sequence set as forth in any one of SEQ ID NO: 19-24; ii. A domain II, wherein a constant region of heavy chain sequence set as forth in SEQ ID NO: 30, or SEQ ID NO: 31; iii. A domain III, comprised a glycine-serine linker sequence set as forth in any one of SEQ ID NO: 33-40, wherein the copy number n=“1-10”; iv. A domain IV, comprising single chain Fv region (scFv) set as forth in SEQ ID NO:10, wherein the scFv fragment binds to an epitope of hepatitis B virus small surface antigen, or hepatitis B virus medium surface antigen, or hepatitis B virus large surface antigen. And (b). the second polypeptide chain comprises, in the N-terminal to C-terminal direction: i. A domain I, wherein a variable light region (VL) binds to an epitope of human CD3 sequence set as forth in any one of SEQ ID NO: 25-29 ii. a domain II, wherein a constant region of light chain sequence set as forth in SEQ ID NO: 32.

In some embodiments, the glycine-serine linker sequence set as forth in SEQ ID NO: 31, wherein the copy number n=“4”.

In some embodiments, the first polypeptide chain comprises an amino acid sequence set as forth in any one of SEQ ID NO: 41-52; the second polypeptide chain comprises an amino acid sequence set as forth in any one of SEQ ID NO: 53-57.

In some embodiments, the first polypeptide chain comprises an amino acid sequence set as forth in any one of SEQ ID NO: 41-46; the second polypeptide chain comprises an amino acid sequence set as forth in SEQ ID NO: 53. In the exemplary embodiment, the bispecific antibody is BsAb2-5-011.

In some embodiments, the first polypeptide chain comprises an amino acid sequence set as forth in any one of SEQ ID NO: 41-46; the second polypeptide chain comprises an amino acid sequence set as forth in SEQ ID NO: 54. In the exemplary embodiment, the bispecific antibody is BsAb2-5-030.

In some embodiments, the first polypeptide chain comprises an amino acid sequence set as forth in any one of SEQ ID NO: 47-52; the second polypeptide chain comprises an amino acid sequence set as forth in SEQ ID NO: 54. In the exemplary embodiment, the bispecific antibody is BsAb2-6-017.

In some embodiments, the first polypeptide chain comprises an amino acid sequence set as forth in any one of SEQ ID NO: 47-52; the second polypeptide chain comprises an amino acid sequence set as forth in SEQ ID NO: 53. In the exemplary embodiment, the bispecific antibodies are BsAb2-6-001, BsAb2-6-006, BsAb2-6-011, and BsAb2-6-016.

The present application further provides a nucleic acid molecule encoding the bispecific antibody described above.

The present application further provides an expression vector comprising the nucleic acid molecule as described above.

The present application further provides a pharmaceutical composition which comprises the bispecific antibody, the nucleic acid molecule, or the vector and a pharmaceutically acceptable excipient, diluent or carrier.

The present application further provides the uses of the bispecific antibody, the nucleic acid molecule, the vector, the host cell, or the pharmaceutical composition in preparation of medicines for preventing or treating HBV infection and other related diseases. In some embodiments, the diseases caused by HBV infection are hepatitis, liver fibrosis, liver cirrhosis, or liver cancer.

The present application further provides a CD3 antibody or antigen-binding fragment thereof. Surprisingly, it is found that the CD3 antibody of the present application has significantly reduced binding affinity for CD3, and the preparation of bispecific antigen-binding molecules with it can overcome the disadvantages of the prior art such as cytokine storm, T-cell depletion, large side effects and narrow dosing window triggered by excessive T-cell activation due to the high affinity of the CD3 antibody.

In some embodiments, the CD3 antibody or antigen-binding fragment thereof, comprising: VH binding domain and VL binding domain; wherein the VH comprises the amino acid sequence that is at least 90% identical to SEQ ID NO: 19, SEQ ID NO: 20, SEQ ID NO: 21, SEQ ID NO: 22, SEQ ID NO: 23, or SEQ ID NO: 24; and/or;
the VL comprises the amino acid sequence that is at least 90% identical to SEQ ID NO: 25, SEQ ID NO: 26, SEQ ID NO: 27, SEQ ID NO: 28, or SEQ ID NO: 29.

In some embodiments, the CD3 antibody or antigen-binding fragment thereof, wherein the amino acid sequence of the VH domain as shown in SEQ ID NO: 19, SEQ ID NO: 20, SEQ ID NO: 21, SEQ ID NO: 22, SEQ ID NO: 23, or SEQ ID NO: 24; and the amino acid sequence of the VL domain as shown in SEQ ID NO: 25.

The present application further provides a bispecific or multispecific antigen-binding molecule comprising the CD3 antibody or an antigen-binding fragment as described above.

In some embodiments, the bispecific antigen-binding molecule comprises a second antigen-binding domain in addition to the CD3 antibody or antigen-binding fragment thereof.

In some embodiments, the second antigen-binding domain comprises a virus-associated antigen-binding domain, or a tumor antigen-binding domain.

In some embodiments, the second antigen binding domain comprises an antibody or fragment thereof capable of binding to the second antigen, TCRs or soluble fragments thereof, receptors or receptor extracellular domains corresponding to any antigen, ligands or ligand extracellular domains, and “derivatives” and “analogs” of the above domains.

In some embodiments, the second antigen binding domain is a hepatitis B surface antigen binding domain, the hepatitis B surface antigen comprising hepatitis B virus small surface antigen, hepatitis B virus medium surface antigen, or hepatitis B virus large surface antigen.

In some embodiments, the hepatitis B surface antigen binding domain is an anti-hepatitis B surface antigen single chain antibody (HBsAg scFv).

The present application further provides a nucleic acid molecule encoding CD3 antibody or an antigen-binding fragment. A nucleic acid molecule encoding the bispecific or multispecific antigen-binding molecule comprising the CD3 antibody or an antigen-binding fragment as described above.

The beneficial effects of the present application relative to the prior art: the present application constructs a format of bispecific antibody that specifically binds to CD3 and HBsAg, with HBsAg scFv fused to the C-terminus of the CD3 antibody (in the form of IgG) heavy chain, which has the property of simultaneously targeting target cells infected with HBV and T cells, bridging T cells to target cells and enhancing the killing of target cells, and also has the property of enhanced neutralization of HBV virus in the blood. The bispecific antibody of the application can eradicate the cells carrying HBV cccDNA. The bispecific antibody of this application can effectively treat HBV-induced infections and liver cancer through killing HBV virus in target cells and blood in multiple directions. Meanwhile, the bispecific antibody of this application is structurally stable, easy to express and purify, and has good pharmacological exploitability.

On the other hand, the present application provides a CD3 antibody or its antigen-binding fragment with significantly reduced affinity for CD3. The preparation of bispecific antigen-binding molecules and multispecific antigen-binding molecules comprising the CD3 antibody or its antigen-binding fragment can overcome the disadvantages of the prior art such as cytokine storm, T-cell depletion, large side effects and narrow dosing window triggered by excessive T-cell transitional activation due to the high activity of the CD3 antibody.

It is to be understood that one, some, or all the properties of the various embodiments described herein may be combined to form other embodiments of the present invention.

The disclosure of all publications, patents, patent applications and published patent applications referred to herein are hereby incorporated herein by reference in their entirety.

### BRIEF DESCRIPTION OF THE DRAWINGS

FIG. 1 depicts useful schematics of four alternative representations of bispecific anti-HBsAg/anti-CD3 antibodies of the invention. It should be noted that the IgG and scFv domain can be switched. (A) and (B) comprise a single chain fragment (scFv) binding to hepatitis B virus surface antigen (HBsAg) fused to the heavy chain N/C terminal of the anti-CD3 IgG antibody, respectively; (C) and (D) comprise a single chain fragment (scFv) binding to human CD3 antigen fused to the heavy chain N/C terminal of the anti-HBsAg IgG antibody, respectively.

FIG. 2 depicts HbsAg binding ELISA of the anti-HbsAg/anti-CD3 bispecific antibodies. BsAb2-5 (A), (B) and bsAb2-6 (C), (D) binds to HbsAg, respectively.

FIG. 3 depicts human CD3ε binding ELISA of the anti-HbsAg/anti-CD3 bispecific antibodies. BsAb2-5 (A), (B) and bsAb2-6 (C), (D) binds to human CD38, respectively.

FIG. 4 depicts rhesus CD3ε binding ELISA of the anti-HbsAg/anti-CD3 bispecific antibodies. BsAb2-5 (A), (B) and bsAb2-6 (C), (D) binds to rhesus CD38, respectively.

FIG. 5 depicts the dose-dependent binding of the anti-HbsAg/anti-CD3 bispecific antibodies to CD3 positive cells and HbsAg-positive cells, respectively. Mean fluorescence intensity for bsAb2-5 (A) and bsAb2-6 (B) binds to CD3⁺ T cells, and bsAb2-6 to HepG2-LMS-660 cells (C).

FIG. 6 depicts the dose-dependent NFAT reporter gene induced by the anti-HbsAg/anti-CD3 bispecific antibodies. BsAb2-5 (A) and bsAb2-6 (B) induced T cell activation was determined by NFAT reporter gene, taken HbsAg uncoated group as negative control.

FIG. 7 depicts the dose-dependent T cell proliferation induced by the anti-HbsAg/anti-CD3 bispecific antibodies. BsAb2-5 (A) and bsAb2-6 (B) induced T cell proliferation was determined by CSFE-labelling flow cytometry analysis.

FIG. 8 depicts the cytokine profile of human PBMC stimulated by the anti-HbsAg/anti-CD3 bispecific antibodies. The cytokine level in the supernatant of bsAb2-6 stimulated human PBMC is measured huTh1/Th2 CBA Kit II (BD, 551809). (A) IFN-γ; (B) TNF-α; (C) IL-10; (D) IL-6; © IL-4; (F) IL-2.

FIG. 9 depicts the anti-HBsAg/anti-CD3 bispecific antibodies re-directed T cell cytotoxicity on HBs+ cell lines. HBs+HepAD38 cells were incubated with bsAb2-6-001, bsAb2-6-006, bsAb2-6-011, bsAb2-6-016, bsAb2-6-017 or bsAb2-5-011, and activated T cells used as effector cells (E:T=3:1).

FIG. 10 depicts the binding affinities of the anti-HBsAg/anti-CD3 bispecific antibodies and recombinant human CD3& as measured by surface plasmon resonance (SPR).

FIG. 11 depicts the binding affinities of the anti-HBsAg/anti-CD3 bispecific antibodies and cynomolgus CD3ε as measured by surface plasmon resonance (SPR).

FIG. 12 depicts the binding affinities of the anti-HBsAg/anti-CD3 bispecific and HBsAg as measured by surface plasmon resonance (SPR).

FIG. 13 depicts SEC-HPLC characterization of the anti-HBsAg/anti-CD3 bispecific antibodies.

FIG. 14 depicts in vitro HBV neutralization potency of the anti-HBsAg/anti-CD3 bispecific antibodies in Huh7-NTCP cells. The newly produced HBeAg in the supernatant, as a marker of a successful HBV infection, is measured by HBeAg ELISA Kit.

FIG. 15 depicts the anti-tumor activities of the anti-HBsAg/anti-CD3 bispecific antibodies on HBs+HepAD38 CDX model. (A) Average tumor volume in mice treated with vehicle, control antibody or bsAb2-6-011 at the indicated dosages. Error bar indicates SEM. (B) Characterization of human leukocytes in HepAD38 xenograft-bearing mice after treated with bsAb2-6-011, or control antibody, stained by human CD45 and determined by flow cytometry at the endpoint. (C) Average bodyweight in mice treated with vehicle, control antibody or bsAb2-6-011 at the indicated dosages. Error bar indicates SEM.

FIG. 16 depicts the pharmacokinetic profile of the anti-HBsAg/anti-CD3 bispecific antibodies in cynomolgus monkeys. (A) Pharmacokinetic profile of bsAb2-6-011 in blood samples at pre-dose and at various time-points after dosing. (B) Bodyweight changes in 5 mpk bsAb2-6-011 treated cynomolgus monkeys.

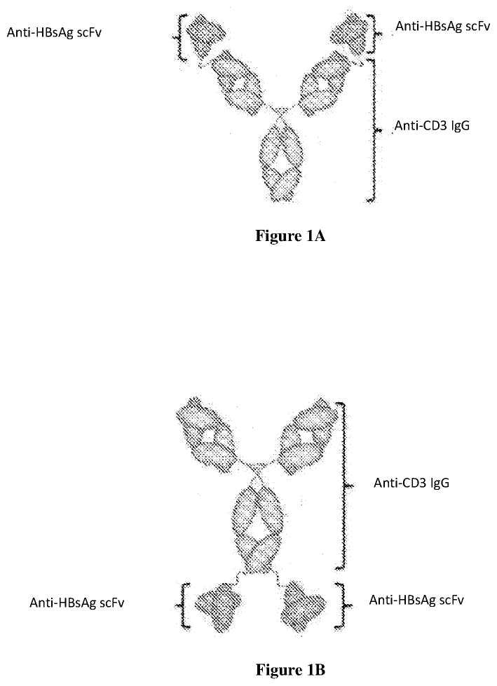

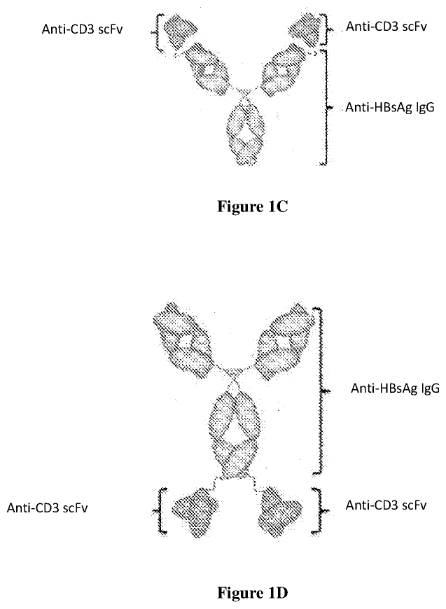

### DETAILED DESCRIPTION OF THE INVENTION

### Definitions

In order to more readily understand the invention, certain technical and scientific terms are specifically defined below. Unless specifically defined elsewhere in this document, all other technical and scientific terms used herein have the meaning commonly understood by one of ordinary skill in the art to which this invention belongs.

The te“m “bispecific antigen-binding molec”le” in this application refers to a molecule capable of binding two different antigens or epitopes, comprising two different antigen-binding domains that are functionally linked or coupled by chemical coupling, gene fusion, non-covalent binding, or other means. The two different antigen-binding domains are selected from one or more of antibodies or fragments thereof, TCRs or soluble fragments thereof, receptors or receptor extracellular domains corresponding to any antigen, ligands or ligand extracellular domains, a“d “derivati”es” a“d “anal”gs” of the above domains.

The te“m “antib”dy” as used in the present invention includes not only complete antibodies, but also fragments, polypeptide sequences, and derivatives and analogues thereof having antigen-binding activity.

The term antigen-binding fragment refers to one or more portions of a full-length antibody, said portion retaining the ability to bind an antigen (e.g., HER2) in competition with the intact antibody for specific binding to the antigen. See generally, Fundamental Immunology, Ch. 7 (Paul, W., ed., 2nd ed., Raven Press, N.Y. (1989), which is incorporated herein by reference in its entirety for all purposes. Antigen-binding fragment can be generated by recombinant DNA technology or by enzymatic or chemical breakage of intact antibodies. In some embodiments, antigen-binding fragment include Fab, Fab′, F(ab′)₂, Fd, Fv, dAb and complementary determining region (CDR) fragments, single chain antibodies (e.g., scFv), chimeric antibodies comprising at least a portion of an antibody sufficient to confer peptide-specific antigen-binding ability. The antigen-binding fragment of the antibody (e.g., the antibody fragment described above) may be obtained from a given antibody (e.g., monoclonal antibody 2E12) using conventional techniques known to those of skill in the art (e.g., recombinant DNA technology or enzymatic or chemical breakage methods) and the antigen-binding fragment of the antibody may be screened for specificity in the same manner as for intact antibodies. The term “Fd fragment” refers to an antibody fragment comprising the VH and CH1 structural domains; the term “Fv fragment” refers to an antibody fragment comprising the VL and VH structural domains of a single arm of the antibody; the term “dAb fragment” refers to an antibody fragment comprising the VH domain (Ward et al., Nature 341:544-546 (1989)); the term “Fab fragment” refers to an antibody fragment consisting of the VL, VH, CL and CH1 structural domains of the antibody; “F(ab′) 2 fragment” refers to an antibody fragment comprising two Fab fragments linked by a disulfide bridge at the hinge region.

The terms “derivative” and “analog” refer to polypeptides that maintain substantially the same biological function or activity as an antibody, a TCR or soluble fragment thereof, an antigen receptor or receptor extracellular domain, an antigen ligand or ligand extracellular domain. The derivative or analog of the present invention may be (i) a polypeptide having one or more conserved or non-conserved amino acid residues (preferably conserved amino acid residues) substituted, and such substituted amino acid residues may or may not be encoded by the genetic code, or (ii) a polypeptide having substituent group in one or more amino acid residues, or (iii) a mature polypeptide with another compound (e.g., a compound that extends the half-life of the polypeptide, such as polyethylene glycol), or (iv) a polypeptide formed by the fusion of additional amino acid sequences to this polypeptide sequence (e.g., a leader sequence, signal peptide, a sequence used to purify this polypeptide, or a fusion protein formed with a 6His tag). According to the teachings herein, these derivatives and analogs are within the scope of what is well known to those skilled in the art.

“Virus-related antigens” in this application refers to antigens related to a variety of known viruses, comprising DNA viruses, RNA viruses, and protein viruses (e.g., prions) classified by genetic material; euvirus and subvirus (viroids, virusoid, prion) classified by viral structure; phages (bacterial virus), plant viruses (e.g., tobacco mosaic virus), animal viruses (e.g., avian influenza virus, smallpox virus, HIV etc.) classified by the host type classification; spherical virus, baculovirus, brick-shaped viruses, coronavirus (e.g., SARS-COV-2), filoviridae, rotavirus, enveloped spherical virus, and virus with a globular head classified by the morphological. They can be antigens presented by the virus itself or antigens formed by the virus after infection of cells.

As used herein, “Hepatitis B virus,” used interchangeably with the term “HBV” refers to the well-known non-cytopathic, liver-tropic DNA virus belonging to the Hepadnaviridae family.

The HBV genome is partially double-stranded, circular DNA with overlapping reading frames. There are four transcripts (that may be referred to herein as “genes” or “open reading frames”) based on size, encoded by the HBV genome. These contain open reading frames called C, X, P, and S. The core protein is coded for by gene C (HBcAg). Hepatitis B e antigen (HBeAg) is produced by proteolytic processing of the pre-core (pre-C) protein. The DNA polymerase is encoded by gene P. Gene S is the gene that codes for the surface antigens (HBsAg). The HBsAg gene is one long open reading frame which contains three in frame “start” (ATG) codons resulting in polypeptides of three different sizes called large, middle, and small S antigens, pre-S1+pre-S2+S, pre-S2+S, or S. Surface antigens in addition to decorating the envelope of HBV, are also part of subviral particles, which are produced at large excess as compared to virion particles, and play a role in immune tolerance and in sequestering anti-HBsAg antibodies, thereby allowing for infectious particles to escape immune detection.

In this application, “tumor antigen” refers to the antigen substance that emerges or is overexpressed during the process of tumorigenesis and development. Tumor antigens are classified according to their specificity: tumor specific antigens and tumor-associated antigens.

Tumor specific antigen (TSA) is a new antigen that is unique to tumor cells or present only in certain tumor cells but not in normal cells. These antigens are also known as tumor specific transplantation antigen (TSTA) or tumor rejection antigen (TRA) because they are confirmed by transplantation of tumors between animals of the same strain. Chemically or physically induced tumor antigens, spontaneous tumor antigens, and virus-induced tumor antigens are mostly in this category.

Tumor-associated antigen (TAA) is an antigen that is not specific to tumor cells but is also present on normal cells and other tissues, except that its level is significantly increased when the cells become cancerous. These antigens exhibit only quantitative changes without strict tumor specificity. For example, embryonic antigens are typical of them.

The CD3ε have the same meaning with CD3 epsilon in this application.

The term “fusion,” when used in connection with a polypeptide or polynucleotide, refers to a form of polypeptide or polynucleotide that does not exist in its natural state, a non-limiting example of which can be achieved by combining polynucleotides or polypeptides that do not normally occur together.

The term “cross-reaction” refers to the ability of the antibody described herein to bind to antigens from different species. For example, the antibody described herein that binds to human CD3 may also bind to CD3 from other species (e.g., cynomolgus monkey CD3). Cross-reactivity may be measured by detecting specific reactivity with purified antigens in binding assays (e.g., SPR, ELISA), or detecting the binding to cells physiologically expressing antigens or the interaction with the function of cells physiologically expressing antigens. Examples of assays known in the art for determining the binding affinity include surface plasmon resonance (e.g., Biacore) or similar techniques (e.g., Kinexa or Octet).

This invention is illustrated by the following non-limiting examples.

In the following examples, the experimental methods of unrecited specific conditions are usually in accordance with conventional conditions such as those in Molecular Cloning by Sambrook et al.: Laboratory Manual (Molecular Cloning-A Laboratory Manual), or according to the methods proposed by manufacturer. The percentages and parts are by weight unless otherwise indicated. Unless otherwise specified, the reagents and materials involved in the text can be obtained commercially or prepared by those skilled in the art according to common knowledge. Any methods and materials similar or equivalent to those described can be used in this application. The preferred implementation methods and materials herein are for exemplary purposes only, but do not limit the content of this application.

### Example 1: Preparation of Anti-HBsAg/Anti-CD3 Bispecific Antibodies Humanization of Anti-CD3 mAb

The Kabat system was used to analyze the sequence of the murine anti-CD3 mAb CDR region. The results showed that the amino acid sequences of HCDR1-3 of the murine anti-CD3 mAb as shown in SEQ ID NO: 11, SEQ ID NO: 12 and SEQ ID NO: 13, respectively; the VH sequence of the murine anti-CD3 mAb as shown in SEQ ID NO: 17; the amino acid sequences of LCDR1-3 of the murine anti-CD3 mAb as shown in SEQ ID NO: 14, SEQ ID NO: 15 and SEQ ID NO: 16, respectively; the VL sequence of the murine anti-CD3 mAb as shown in SEQ ID NO: 18.

To improve the humanization of the murine anti-CD3 mAb by chimerizing the backbone regions of the heavy and light chain variable regions of human immunoglobulins.

Comparing the protein sequence of the murine anti-CD3 mAb heavy chain variable region with that of the known human germline immunoglobulin heavy chain, it was confirmed that the murine anti-CD3 mAb heavy chain variable region has 79% and 74% homology with the human germline IGHV3-73*01 and IGHV3-23*05, respectively. They can be used as a chimeric receptor (acceptor) for the murine anti-CD3 mAb heavy chain CDR.

Comparing the protein sequence of the mouse-derived anti-CD3 mAb light chain variable region with that of the known human germline immunoglobulin light chain, it was confirmed that the murine anti-CD3 mAb light chain variable region is 60.0% and 57% homologous to the human germline IGLV7-43*01 and IGLV8-61*01, respectively. They can be used as a chimeric receptor (acceptor) for the murine anti-CD3 mAb light chain CDR.

To improve the affinity of the murine anti-CD3 mAb antibody, revertant mutations can be made to some key amino acids in the backbone regions of the chimeric heavy and light chains, respectively. SEQ ID NO: 19 (Humanized CD3 mAb VH1), SEQ ID NO: 20 (Humanized CD3 mAb VH2), SEQ ID NO: 21 (Humanized CD3 mAb VH3), SEQ ID NO: 22 (Humanized CD3 mAb VH4), SEQ ID NO: 23 (Humanized CD3 mAb VH5), SEQ ID NO: 24 (Humanized CD3 mAb VH6) are the amino acid sequences of the heavy chain variable region of humanized anti-CD3 mAb; SEQ ID NO: 25 (Humanized CD3 mAb VL1), SEQ ID NO: 26 (Humanized CD3 mAb VL2), SEQ ID NO: 27 (Humanized CD3 mAb VL3), SEQ ID NO: 28 (Humanized CD3 mAb VL4), SEQ ID NO: 29 (Humanized CD3 mAb VL5) are the amino acid sequences of the light chain variable region of humanized anti-CD3 mAb.

### Clone and Production of Anti-HBsAg/Anti-CD3 Bispecific Antibodies

DNA sequences encoding the bispecific anti-HBsAg/anti-CD3 antibodies were synthesized by Genewiz and subcloned into pcDNA3.1 vector via restriction enzyme site XbaI & NheI, respectively. The amino acid sequences encoded by the DNA are shown in Table 1.

The BsAb2-5 series of bispecific antibodies are anti-HBsAg antibody C8 scFv (SEQ ID NO: 10) fused to the N-terminal of the heavy chain of the murine anti-CD3 mAb (the bispecific antibody No. BsAb2-5) or humanized anti-CD3 mAb (the bispecific antibody No. BsAb2-5-001˜BsAb2-5-030). The BsAb2-6 series of bispecific antibodies are anti-HBsAg antibody C8 scFv (SEQ ID NO: 10) fused to the C-terminus of the heavy chain of the murine anti-CD3 Ab (the bispecific antibody No. BsAb2-6) or humanized anti-CD3 mAb (the bispecific c antibody No. BsAb2-6-001˜BsAb2-6-030) via a linker. Specifically, the corresponding light and heavy chain sequences of each bispecific antibody and the combination form of humanized VH and VL of the anti-CD3 antibody are shown in Table 1. For example, BsAb2-5-001, the heavy chain is arranged from N-terminal to C-terminal as C8 scFv, VH1-CH1-Fc, and the light chain is VL1-CL; and finally two identical heavy and light chains form a homodimer through disulfide bonds; wherein C8 scFv (SEQ ID NO: 10) is a single-chain antibody to HBsAg; VH1 and VL1 are the heavy chain variable region variant 1 and light chain variable region variant 1 of the humanized mouse-derived anti-CD3 mAb, respectively. BsAb2-6-012, the heavy chain is arranged from N-terminal to C-terminal in the following order: VH3-CH1-Fc, and C8 scFv, and the light chain is VL2-CL; wherein C8 scFv (SEQ ID NO: 10) is a single chain antibody against HBsAg; VH3 and VL2 are the heavy chain variable region variant 3 and light chain variable region variant 2 of the humanized mouse-derived antibody anti-CD3 mAb, respectively.

TABLE 1
The light and heavy chain sequences of the corresponding bispecific antibodies
and the combination form of the humanized VH and VL of anti-CD3 antibodies.

| The combination form | | Sequence | Sequence | | Sequence | Sequence |
|---|---|---|---|---|---|---|
| of the humanized VH | | of HC | of LC | | of HC | of LC |
| and VL of anti-CD3 | Molecular | (SEQ ID | (SEQ ID | Molecular | (SEQ ID | (SEQ ID |
| antibodies | name | NO:) | NO:) | name | NO:) | NO:) |
| VH1VL1 | BsAb2-5-001 | 41 | 53 | BsAb2-6-001 | 47 | 53 |
| VH1VL2 | BsAb2-5-002 | 41 | 54 | BsAb2-6-002 | 47 | 54 |
| VH1VL3 | BsAb2-5-003 | 41 | 55 | BsAb2-6-003 | 47 | 55 |
| VH1VL4 | BsAb2-5-004 | 41 | 56 | BsAb2-6-004 | 47 | 56 |
| VH1VL5 | BsAb2-5-005 | 41 | 57 | BsAb-2-6-005 | 47 | 57 |
| VH2VL1 | BsAb2-5-006 | 42 | 53 | BsAb-2-6-006 | 48 | 53 |
| VH2VL2 | BsAb2-5-007 | 42 | 54 | BsAb2-6-007 | 48 | 54 |
| VH2VL3 | BsAb2-5-008 | 42 | 55 | BsAb2-6-008 | 48 | 55 |
| VH2VL4 | BsAb2-5-009 | 42 | 56 | BsAb2-6-009 | 48 | 56 |
| VH2VL5 | BsAb2-5-010 | 42 | 57 | BsAb2-6-010 | 48 | 57 |
| VH3VL1 | BsAb2-5-011 | 43 | 53 | BsAb2-6-011 | 49 | 53 |
| VH3VL2 | BsAb2-5-012 | 43 | 54 | BsAb2-6-012 | 49 | 54 |
| VH3VL3 | BsAb2-5-013 | 43 | 55 | BsAb-2-6-013 | 49 | 55 |
| VH3VL4 | BsAb2-5-014 | 43 | 56 | BsAb2-6-014 | 49 | 56 |
| VH3VL5 | BsAb2-5-015 | 43 | 57 | BsAb2-6-015 | 49 | 57 |
| VH4VL1 | BsAb2-5-016 | 44 | 53 | BsAb-2-6-016 | 50 | 53 |
| VH4VL2 | BsAb2-5-017 | 44 | 54 | BsAb2-6-017 | 50 | 54 |
| VH4VL3 | BsAb2-5-018 | 44 | 55 | BsAb2-6-018 | 50 | 55 |
| VH4VLA | BsAb2-5-019 | 44 | 56 | BsAb2-6-019 | 50 | 56 |
| VH4VL5 | BsAb2-5-020 | 44 | 57 | BsAb2-6-020 | 50 | 57 |
| VH5VL1 | BsAb2-5-021 | 45 | 53 | BsAb2-6-021 | 51 | 53 |
| VH5VL2 | BsAb2-5-022 | 45 | 54 | BsAb2-6-022 | 51 | 54 |
| VH5VL3 | BsAb2-5-023 | 45 | 55 | BsAb2-6-023 | 51 | 55 |
| VH5VL4 | BsAb2-5-024 | 45 | 56 | BsAb2-6-024 | 51 | 56 |
| VH5VL5 | BsAb2-5-025 | 45 | 57 | BsAb2-6-025 | 51 | 57 |
| VH6VL1 | BsAb2-5-026 | 46 | 53 | BsAb2-6-026 | 52 | 53 |
| VH6VL2 | BsAb2-5-027 | 46 | 54 | BsAb2-6-027 | 52 | 54 |
| VH6VL3 | BsAb2-5-028 | 46 | 55 | BsAb2-6-028 | 52 | 55 |
| VH6VLA | BsAb2-5-029 | 46 | 56 | BsAb2-6-029 | 52 | 56 |
| VH6VL5 | BsAb2-5-030 | 46 | 57 | BsAb2-6-030 | 52 | 57 |

### Expression in Expi293 Cells

The bispecific antibodies are expressed by transient co-transfection of the respective expression plasmids (e.g., encoding the heavy and modified heavy chain, as well as the corresponding light and modified light chain at a ratio of 1:1) using the Expi293 system (Invitrogen, #A14635CN) according to the manufacturer's instruction. Briefly, Expi293 cells growing in suspension either in a shake flask or in a stirred fermenter in serum free Expi293 expression medium is transfected with a mix of the two expression plasmids and Expi293 fectin. For 250 mL shake flask Expi293 cells are seeded at a density of 1E6 cells/mL in 100 mL and incubated at 125 rpm, 8% CO₂. The day after the cells is transfected at a cell density of 2.5E6 cells/mL with 10 mL mix of A) 5 mL Opti-MEM with 100 μg total plasmid DNA (1 μg/mL, 50 μg heavy chain plasmid and the corresponding light chain in an equimolar ratio) and B) 5 mL Opti-MEM with 260 μL Expi293 fectin (2.6 μL/mL). According to the instruction, enhancer1 and 2 are added the day after transfecting day. The supernatant containing the secreted antibody is harvested after 5 days and antibodies are either directly purified from the supernatant, or the supernatant is frozen and stored.

### Purification

Proteins were purified from filtered cell culture supernatants referring to standards protocols. In brief, supernatant of BsAb2-5 and BsAb2-6 constructs were applied to a one-step Protein A-affinity chromatography (equilibrating buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. Aggregated protein was separated from monomeric antibodies by size exclusion chromatography (Superdex200, GE Healthcare) in PBS or in 20 mM Histidine, 50 mM NaCl at pH 5.5. Monomeric molecule fractions can be pooled, concentrated (if required) using MILLIPORE Amicon Ultra (30 MWCO) centrifugal concentrator, frozen and stored at −20° C. or −80° C. Part of the sample can be provided for subsequent protein analytics characterization e.g., by SDS-PAGE or size exclusion chromatography (SEC) or mass spectrometry.

### Protein Determination

The concentration of purified antibodies is determined by the optical density (OD) at 280 nm, using the molar extinction coefficient calculated based on the amino acid sequence according to Pace et al., Protein Science, 1995, 4, 2411-1423.

### Example 2 Binding Properties of Anti-HBsAg/Anti-CD3 Bispecific Antibodies

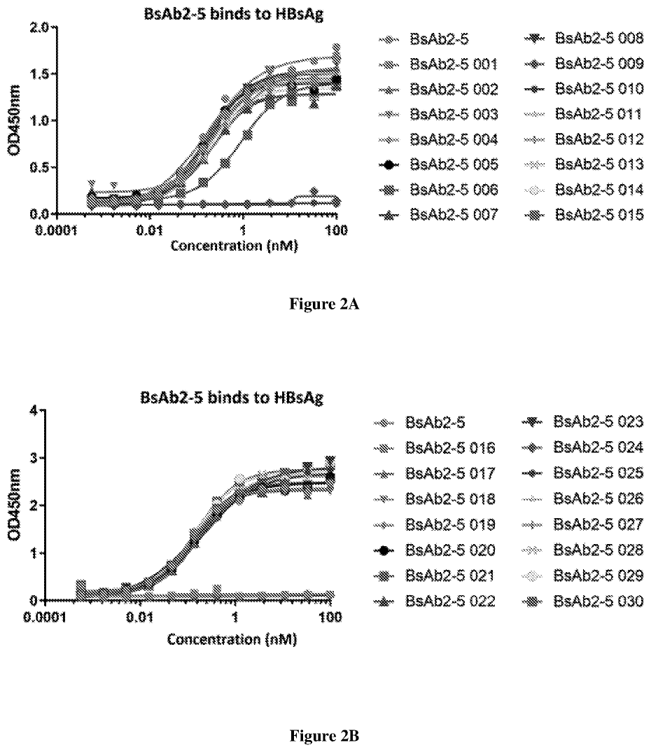

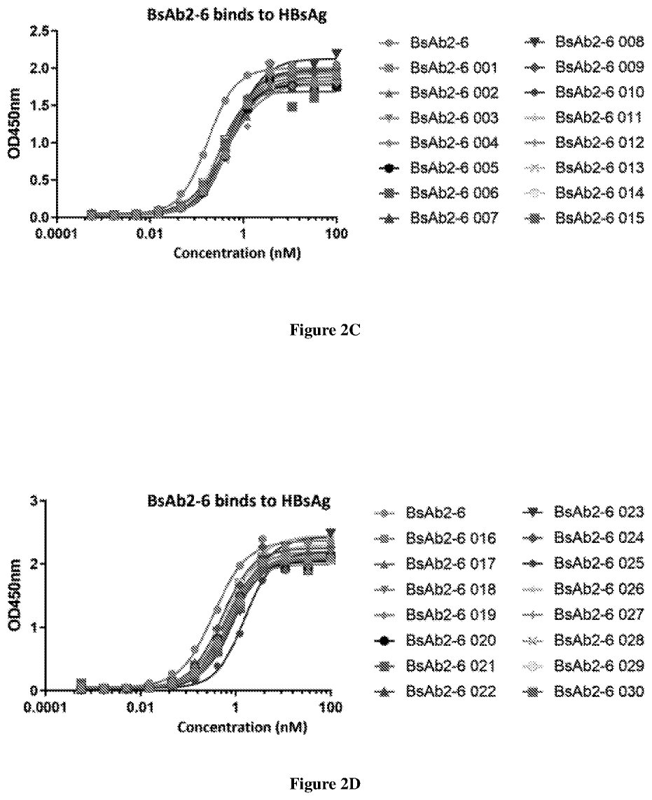

Anti-HBsAg/anti-CD3 bispecific antibodies generated in Example 1 are analyzed by standard ELISA techniques for their binding properties to HBsAg, human CD3E, and rhesus CD3ε recombinant protein, respectively. FIGS. 2A to 2D and Table 2 show that the humanized anti-CD3 mAb derived bsAb2-5 and bsAb2-6 constructs had strong binding to recombinant HBsAg.

TABLE 2
Binding ability of the bispecific antibodies to human CD3ε, rhesus
CD3ε and HBsAg recombinant proteins (EC50, nM).

| | EC50 (nM) | | |
|---|---|---|---|
| Molecular | | Human | rhesus |
| name | HBsAg | CD3ε | CD3ε |
| BsAb2-5-001 | 0.23 | 0.89 | 0.72 |
| BsAb2-5-002 | 0.16 | 0.31 | 0.30 |
| BsAb2-5-003 | 0.21 | 0.30 | 0.34 |
| BsAb2-5-004 | 0.36 | NA | NA |
| BsAb2-5-005 | 0.15 | 0.26 | 0.31 |
| BsAb2-5-006 | 0.90 | 2.72 | 2.12 |
| BsAb2-5-007 | 0.23 | 0.29 | 0.40 |
| BsAb2-5-008 | 0.19 | 0.37 | 0.24 |
| BsAb2-5-009 | NA | NA | ~11.59 |
| BsAb2-5-010 | NA | NA | NA |
| BsAb2-5-011 | 0.20 | 1.85 | 1.99 |
| BsAb2-5-012 | 0.16 | 0.32 | 0.24 |
| BsAb2-5-013 | 0.17 | 0.33 | 0.26 |
| BsAb2-5-014 | 0.20 | NA | NA |
| BsAb2-5-015 | 0.17 | 0.27 | 0.27 |
| BsAb2-5-016 | NA | NA | NA |
| BsAb2-5-017 | NA | NA | NA |
| BsAb2-5-018 | 0.12 | 0.33 | 0.24 |
| BsAb2-5-019 | NA | NA | NA |
| BsAb2-5-020 | 0.16 | 0.28 | 0.26 |
| BsAb2-5-021 | 0.14 | 7.54 | 3.88 |
| BsAb2-5-022 | 0.16 | 0.28 | 0.31 |
| BsAb2-5-023 | 0.18 | 0.23 | 0.22 |
| BsAb2-5-024 | NA | NA | NA |
| BsAb2-5-025 | 0.20 | 0.40 | 0.25 |
| BsAb2-5-026 | 0.18 | 6.18 | 2.09 |
| BsAb2-5-027 | 0.16 | 0.49 | 0.28 |
| BsAb2-5-028 | 0.19 | 0.50 | 0.31 |
| BsAb2-5-029 | NA | NA | NA |
| BsAb2-5-030 | 0.15 | 0.41 | 0.23 |
| BsAb2-6-001 | 0.50 | 0.53 | 0.29 |
| BsAb2-6-002 | 0.46 | 0.30 | 0.14 |
| BsAb2-6-003 | 0.47 | 0.34 | 0.17 |
| BsAb2-6-004 | 0.43 | NA | NA |
| BsAb2-6-005 | 0.45 | 0.33 | 0.18 |
| BsAb2-6-006 | 0.31 | 1.04 | 0.76 |
| BsAb2-6-007 | 0.42 | 0.25 | 0.19 |
| BsAb2-6-008 | 0.57 | 0.36 | 0.28 |
| BsAb2-6-009 | 0.45 | NA | 35.20 |
| BsAb2-6-010 | 0.46 | 0.30 | 0.23 |
| BsAb2-6-011 | 0.44 | 0.66 | 0.34 |
| BsAb2-6-012 | 0.45 | 0.32 | 0.24 |
| BsAb2-6-013 | 0.47 | 0.33 | 0.26 |
| BsAb2-6-014 | 0.31 | NA | NA |
| BsAb2-6-015 | 0.33 | 0.19 | 0.18 |
| BsAb2-6-016 | 0.71 | 0.80 | 0.36 |
| BsAb2-6-017 | 0.60 | 0.40 | 0.15 |
| BsAb2-6-018 | 0.73 | 0.50 | 0.16 |
| BsAb2-6-019 | 0.53 | NA | NA |
| BsAb2-6-020 | 0.56 | 0.32 | 0.12 |
| BsAb2-6-021 | 0.80 | 1.70 | 0.83 |
| BsAb2-6-022 | 0.77 | 0.41 | 0.23 |
| BsAb-2-6-023 | 1.09 | 1.09 | 0.26 |
| BsAb-2-6-024 | 0.51 | NA | NA |
| BsAb2-6-025 | 1.50 | 1.50 | 0.50 |
| BsAb2-6-026 | 0.69 | 0.69 | 0.63 |
| BsAb2-6-027 | 0.72 | 0.72 | 0.21 |
| BsAb2-6-028 | 0.83 | 0.83 | 0.21 |
| BsAb2-6-029 | 0.48 | 0.48 | NA |
| BsAb2-6-030 | 0.48 | 0.48 | 0.13 |

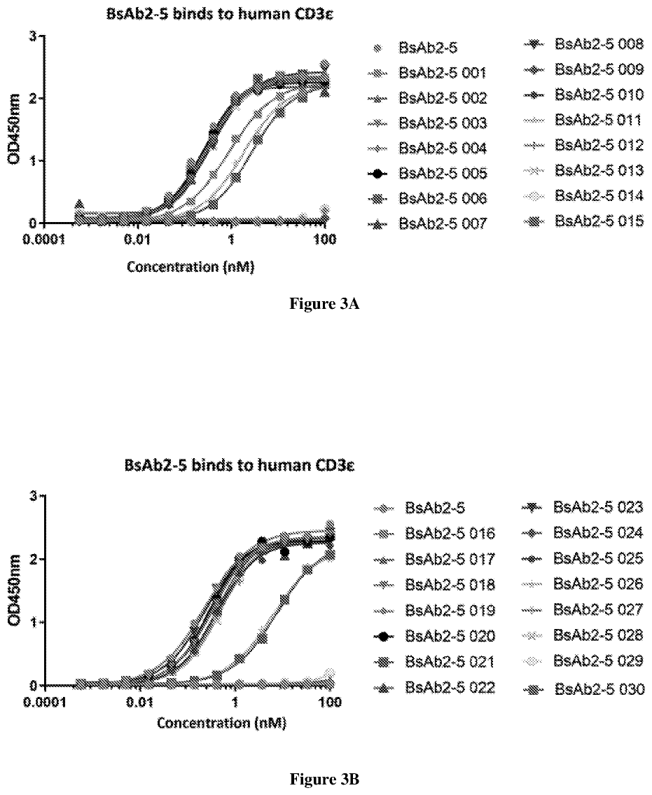

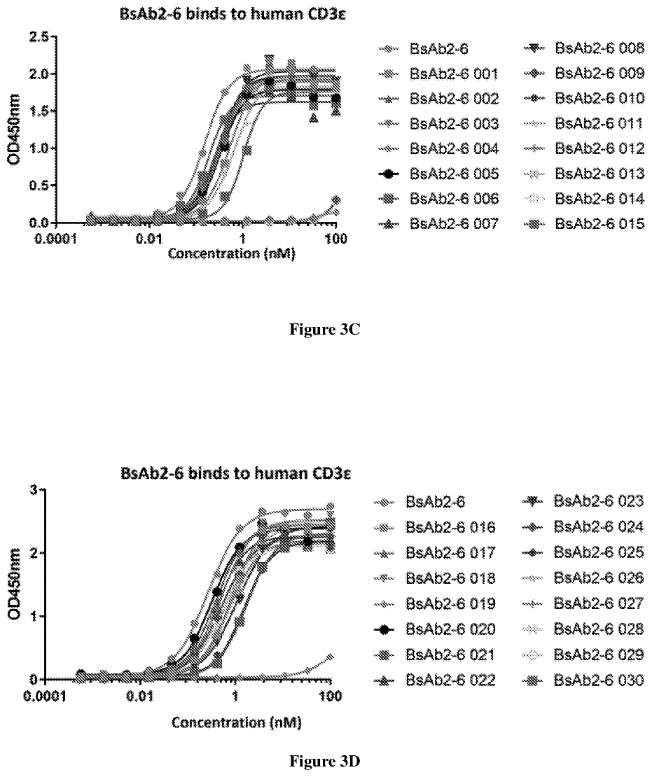

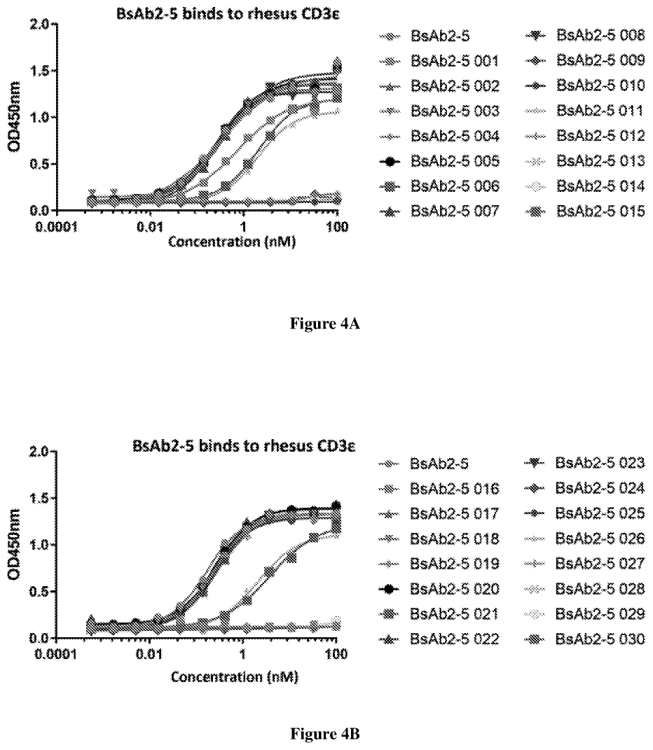

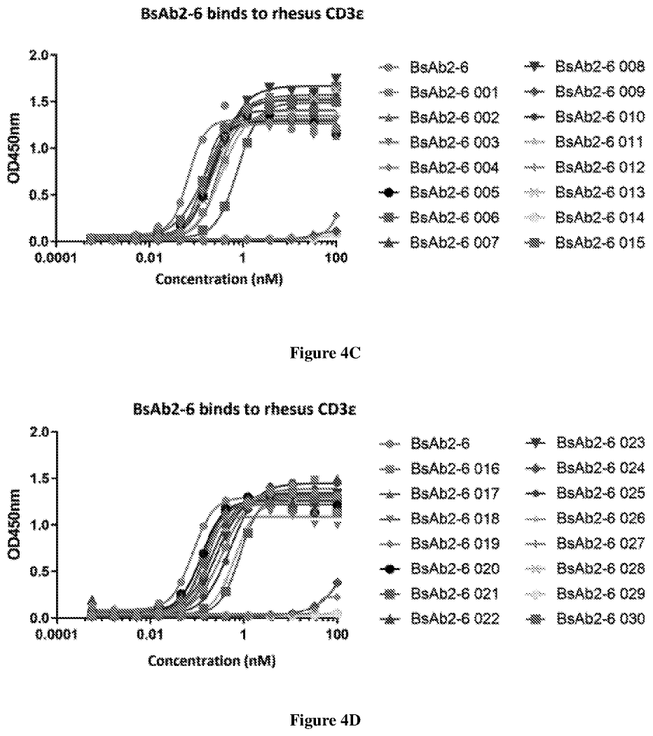

The binding properties of BsAb2-5 and BsAb2-6 constructs to human CD3ε and rhesus CD3ε are illustrated by FIG. 3, FIG. 4 and Table 2. Compared to other molecules, the bispecific antibodies derived in combination with VL1 of bsAb2-5-001, bsAb2-5-006, bsAb2-5-011 bsAb2-5-016, bsAb2-5-021, bsAb2-5-026, bsAb2-6-001, bsAb2-6-006, bsAb2-6-011, bsAb2-6-016, bsAb2-6-021, and bsAb2-6-026 molecules showed a significant decrease in binding ability to human and rhesus CD38 recombinant proteins. While, the bispecific antibodies derived in combination with VL4 of bsAb2-5-004, bsAb2-5-009, bsAb2-5-014, bsAb2-5-019, bsAb2-5-024, bsAb2-5-029, bsAb2-6-004, bsAb2-6-009, bsAb2-6-014, bsAb2-6-019, BsAb2-6-024, and bsAb2-6-029 molecules showed no binding cross with to human and rhesus CD3E.

Anti-HBsAg/anti-CD3 T cell bispecific antibodies generated in Example 1 are analyzed by flow cytometry for their binding properties to CD3 expressed on human leukemic T cell line Jurkat and HBsAg on the surface of human hepatoma cell line HepG2-LMS-660. Exactly, two kinds of target cells are harvested, counted, and adjusted to 2E6 cell per mL in FACS buffer (PBS with 2% BSA). 50 μL of the cell suspension are further aliquoted per well into U-bottom 96-well plate together with 50 μL FACS buffer diluted anti-HBsAg/anti-CD3 bispecific antibodies or human IgG isotype as negative control, final concentrating from 300 nM to 0.017 nM. After 60-min incubation at room temperature, cells are centrifuged (3 min, 300×g) and washed twice with 150 μL/well FACS buffer. Cells are then resuspended with 100 μL 5000-fold diluted APC labeled anti-human Fc secondary antibody (Jackson, #615-605-214) and incubated for 30-min at room temperature, followed by three times washing with 150 μL/well FACS. The cells are finally harvested by centrifugation, resuspended by 100 μL FACS buffer, and applied to flow cytometry (Beckman) and data processed using FlowJo and GraphPad Prism.

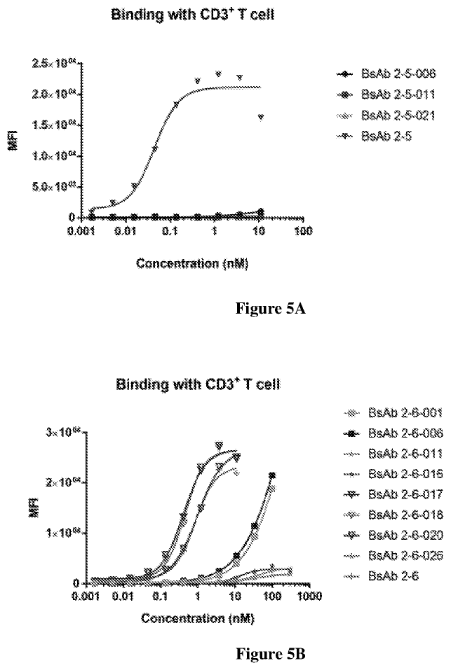

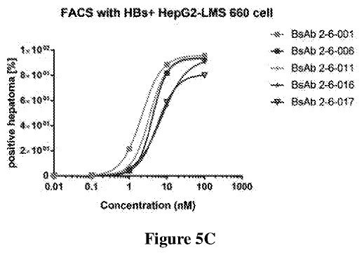

The binding properties of BsAb2-5 and BsAb2-6 constructs to CD3ε expressed on the surface of Jurkat in flow cytometry are shown in FIGS. 5A and 5B. All the tested bsAb2-6 constructs could bind to CD3ε on the surface of Jurkat cells. Clone bsAb2-6, bsAb2-6-017, bsAb2-6-018 and bsAb2-6-020 had the best binding properties of all the tested variants while the bispecific antibodies derived in combination with VL1 of BsAb2-6-001, 006, 011, 016, 021 and 026 had a dramatically reduced property compared to bsAb2-6-017. Similarly, bsAb2-5-006, bsAb2-5-011 and bsAb2-5-021 hardly binded to hCD3ε compared to parental bsAb2-5. This result was consistent with the binding properties with hCD38 recombinant protein in Table 2.

FIG. 5C shows the binding properties of BsAb2-5 and BsAb2-6 constructs to HBsAg on the surface of HepG2-LMS-660 cells. All the tested BsAb2-5 and BsAb2-6 constructs could bind to HBsAg on the surface of HepG2-LMS-660 cells with similar binding affinity. The results showed that the binding affinity with HBsAg is not changed in BsAb2-5 and BsAb2-6 constructs.

### Example 3 T Cell Activation Induced by Anti-HBsAg/Anti-CD3 Bispecific Antibodies

NFAT reporter (luc) Jurkat cells purchased from BPS Bioscience is stably expressing a firefly luciferase gene under the control of the NFAT response element integrated into Jurkat cells and can be used to evaluate the functionalities of anti-HBsAg/anti-CD3 bispecific antibodies, together with the non-specific activation of NFAT signaling pathway. Exactly, NFAT reporter-luc cells are thawed and grown in RPMI-1640 media supplemented with 10% fetal bovine serum with 1 mg/mL geneticin. For high throughput screening, 0.025 million Jurkat cells per well in 20 μL RPMI-1640 complete medium is seeded into 2 μg/mL HBsAg precoated transparent-bottom white 384-well plate and added with 3-fold diluted anti-HBsAg/anti-CD3 bispecific antibodies in 10 μL RPMI-1640 medium, final concentrations are from 27 nM to 0.032 nM. To ensure the anti-HBsAg/anti-CD3 bispecific antibodies induce NFAT reporter gene strictly cross-linked with HBsAg, well containing 27 nM anti-HBsAg/anti-CD3 but without HBsAg precoated well is taken as negative control. After 6 hrs incubation at 37° C., 5% CO₂, 15 μL OneGlo luciferase substrate (Promega #E6120) is added to each well. Luminescence is measured on an TECAN INFINITE M PLEX plate reader after 5-minute incubation and readout data is processed using GraphPad Prism.

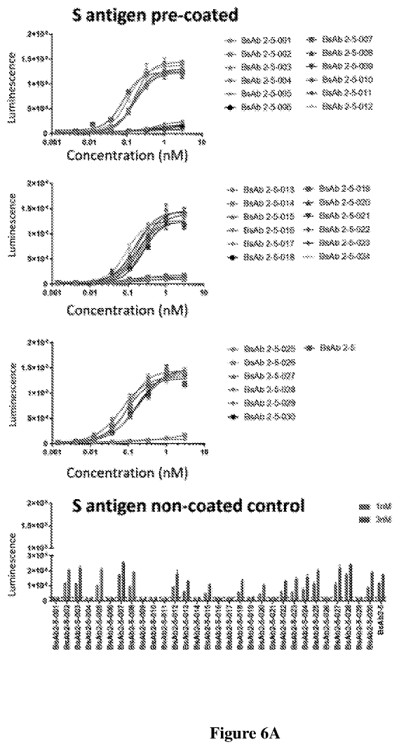

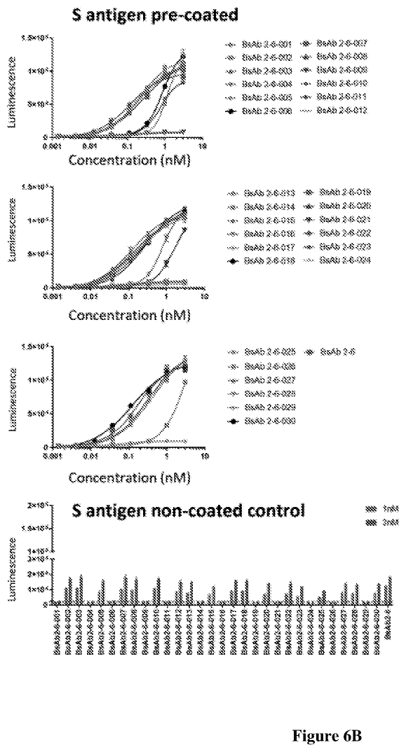

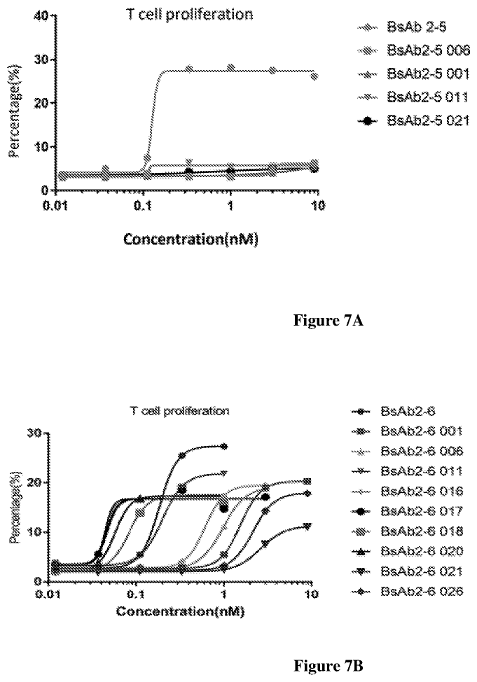

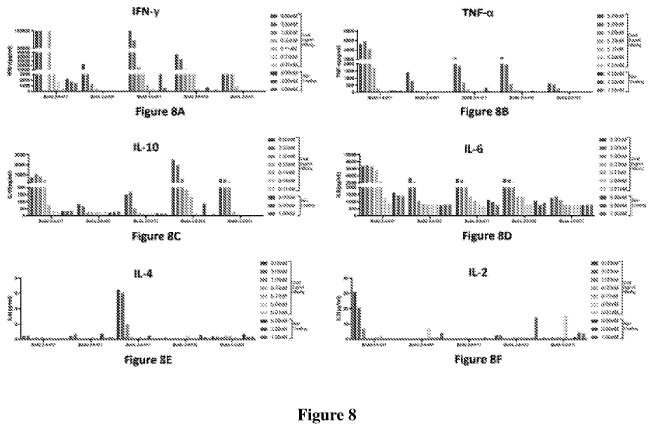

FIG. 6A and FIG. 6B show that the anti-HBsAg/anti-CD3 bispecific antibodies induced T-cell activation only in the presence of HBsAg antigen. The BsAb2-6-017 had the strongest ability to activate T cells, and the VL1 combination-derived BsAb2-6-001, BsAb2-6-006, BsAb2-6-011, BsAb2-6-016, BsAb2-6-021, and BsAb2-6-026 T cells had much weaker activation ability with their EC₅₀ values differed from 5 to 10-fold. Similarly, paretal bsAb2-5 had comparable potency on T cell activation to that of BsAb2-6-017, while BsAb2-5-001, BsAb2-5-006, BsAb2-5-011, BsAb2-5-016, BsAb2-5-021, and BsAb2-5-026 in the same structure failed to induce T cell activation.

### Example 4: T Cell Proliferation Induced by Anti-HBsAg/Anti-CD3 Bispecific Antibodies

The anti-HBsAg/anti-CD3 bispecific antibodies generated in Example 1 are analyzed by flow cytometry for their potency to induce proliferation of human primary T cells in the presence of HBsAg. Exactly, U-bottom 96-well plate is precoated with 2 μg/mL HBsAg recombinant protein overnight at 4° C. before cell seeding. Frozen human PBMC is quickly thawed in 37° C. water-bath, then transferred to pre-warmed RPMI-1640 medium at the density of 1E6 cells/mL and stained with 1 uM CFSE at room temperature for 5 min, protecting from lighting. The staining reaction is then stopped by addition of equal volume of RPMI-1640 medium supplemented with 10% FBS, and further washed once with pre-warmed complete RPMI-1640 medium to remove free CFSE. CFSE labeled total PBMC are then suspended into 2E6 cells/mL in RPMI-1640 complete medium. Meanwhile, 2 μg/mL HBsAg coating solution is removed from 96-well plate followed by three times PBS washing. 50 μL of cell suspension is added into each well together with 5-fold diluted anti-HBsAg/anti-CD3 bispecific antibodies in 50 μL RPMI-1640 medium, final concentrating from 50 nM to 0.016 nM. To exclude the non-specific T cell proliferation induced by anti-HBsAg/anti-CD3 bispecific antibodies without HBs antigen, 50 nM anti-HBsAg/anti-CD3 and CFSE labeled PBMC but without HBsAg coating well is taken as negative control. After 3 days incubation at 37° C., 5% CO₂, cells are collected by centrifugation (5 min, 300×g) and washed twice with 150 μL per well of FACS wash buffer. Total cells are analyzed by flow cytometry (Beckman) and FlowJo software. Percentage level of total T cells proliferating is determined by all CFSE dilution peaks.

FIGS. 7A to 7C illustrate T cell proliferation induced by the anti-HBsAg/anti-CD3 bispecific antibodies in the presence of HBsAg antigen. The BsAb2-6-017 induced the strongest T cell proliferation, and the VL1 combination derived BsAb2-6-001. BsAb2-6-006, BsAb2-6-011, BsAb2-6-016, BsAb2-6-021, and BsAb2-6-026 showed weaker induction on T cell proliferation. Similarly, parental bsAb2-5 showed comparable potency on T cell proliferation to that of BsAb2-6-017, while the same structure of BsAb2-5-001. BsAb2-5-006, BsAb2-5-011, BsAb2-5-021 failed to induce T cell proliferation.

### Example 5: Cytokine Profile of T Cells Induced by Anti-HBsAg/Anti-CD3 Bispecific Antibodies

The anti-HBsAg/anti-CD3 T cell bispecific antibodies generated in Example 1 are analyzed for their potency to induce cytokine secretion of human primary T cells with or without of HBsAg. Exactly, U-bottom 96-well plate is precoated with 2 μg/mL HBsAg recombinant protein overnight at 4° C. before cell seeding. Frozen human PBMC is quickly thawed in 37° C. water-bath, then transferred to pre-warmed RPMI-1640 complete medium at the density of 1 million cells per mL. 100 μL PBMC are seeded into 2 μg/mL HBsAg coated 96-well plate after three times PBS washing and together addition with 5-fold diluted anti-HBsAg/anti-CD3 bispecific antibodies in 10 μL RPMI-1640 medium, final concentrating from 50 nM to 0.016 nM. To exclude the non-specific cytokine secretion induced by anti-HBsAg/anti-CD3 bispecific antibodies without HBs antigen, 50 nM anti-HBsAg/anti-CD3 containing and primary PBMC but without HBsAg coating well is taken as negative control. After 3 days incubation at 37° C., 5% CO₂, supernatant is collected by centrifugation (20 min, 3000×g) and transferred into a new 96-well plate for CBA analysis according to the manufacture's instruction (BD, #551809). IFN-γ, TNF-α, IL-2, IL-4, IL-6 and IL-10 produced by human Th1/Th2 cells are determined and readout data is processed using GraphPad Prism.

FIGS. 8A to 8F illustrate the successful induction of T cell activation by the anti-HBsAg/anti-CD3 bispecific antibodies in the presence of HBsAg antigen and consequently the secretion of Th1/Th2 class cytokines (IFN-γ, IL-2, IL-4, IL-6, IL-10, TNF-α.). BsAb2-6-017 induced the stronger T cells activation with more cytokine secretion, while the VL1 combination derived BsAb2-6-001, BsAb2-6-011, and BsAb2-6-016 induced the weaker T cell activation with lesser cytokine secretion. Under the same condition, BsAb2-5-001 failed to induce T cells to secrete the relevant cytokines.

### Example 6 Cytotoxicity of T Cells Induced by Anti-HBsAg/Anti-CD3 Bispecific Antibodies

The anti-HBsAg/anti-CD3 bispecific antibodies generated in Example 1 are analyzed for their potency to induce cytotoxic T cell mediated lysis on HBsAg positive HepAD38 cell line depending on HBsAg crosslinking. Exactly, E-Plate 96 plates were coated with collagen (1:30 dilution) for 30 min at 37° C., then added with M10 complete medium and applied to xCelligence Real Time Cell Analyzer (RTCA) to set the baseline of the assay program. HepAD38 cell was harvested and resuspended with M10 complete medium at the density of 1×10⁵ cells/mL, 100 μL/well target cells (1×10⁴ cells/well) were added to E-Plate 96, resting for 5 min, then the cell growth curve was monitored continuously on RTCA overnight. The next day, human peripheral blood total T cells induced 7 days by 400 IU/mL human IL2 were collected as effector cells, and the cell density was adjusted to 3×10⁵ cells/mL; meanwhile, 3-fold diluted anti-HBsAg/anti-CD3 bispecific antibodies (concentration range 300 nM˜1.23 nM) in M10 complete medium was prepared; 50 μL effector T cells (effector cell: target cell ratio=3:1) together with 50 μL of gradient-diluted anti-HBsAg/anti-CD3 bispecific antibody were mixed and added to HepAD38 culturing well. To detect non-specific killing of HepAD38 cells by effector T cells, additional effector T cells and HepAD38 cells but without antibody group were used as negative control. After 72 hrs continuous monitoring, cell growth curve (Plot) was exported.

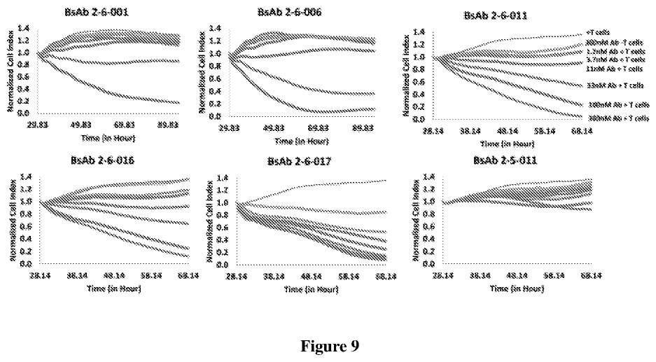

FIG. 9 shows that exemplary anti-HBsAg/anti-CD3 bispecific antibodies induced T cells to kill HBsAg-positive hepatocellular carcinoma cells HepAD38. The results showed that HBsAg/CD3 bispecific antibodies exert differential cytolytic effect on HepAD38 cells in a dose-dependent manner. The bsAb2-6-017 induced the strongest T cell cytolytic effect on HepAD38 at the dose of 33 nM. BsAb2-6-011 and bsAb2-6-016 stimulated T cell killing at the dose of 300 nM, whereas bsAb2-6-001 and bsAb2-6-006 also showed incomplete killing at the same condition. However, bsAb2-5-001 failed to induce the killing effect of T cells on HepAD38 cells under the same condition.

### Example 7 Surface Plasmon Resonance (SPR) Characterization of Anti-HBsAg/Anti-CD3 Bispecific Antibodies

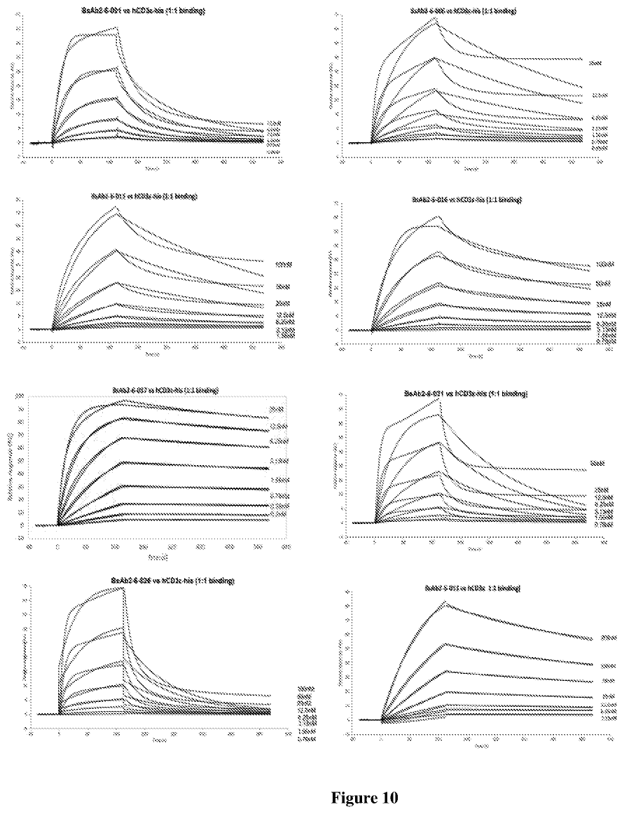

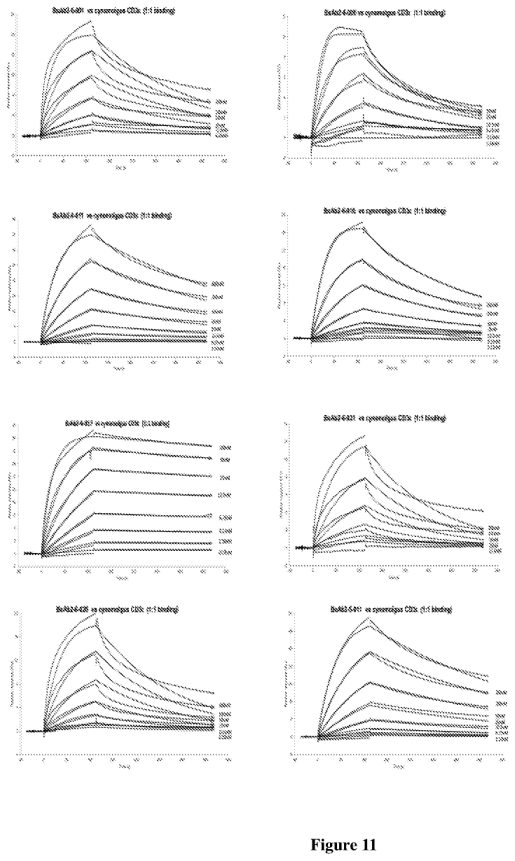

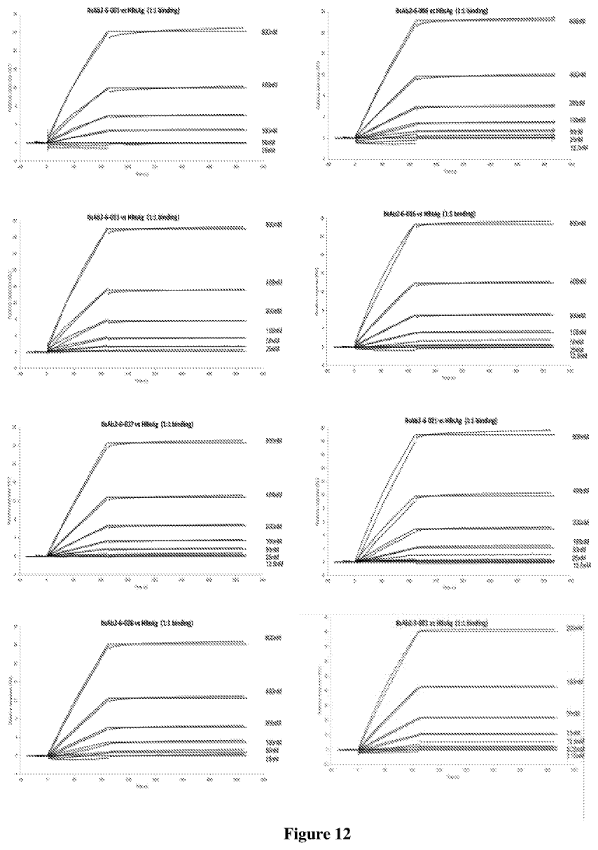

Binding affinity of anti-HBsAg/anti-CD3 constructs to HBsAg and human/cynomolgus CD3ε is characterized by SPR using Biacore T800 (GE Healthcare). Specifically, the anti-HBsAg/anti-CD3 bispecific antibodies were immobilized on protein A chip at a flow rate of 10 μL/min, and gradient dilution of huCD3ε, cynoCD3ε, or HBsAg antigen was used as the mobile phase. The binding condition was 10 μL/min×180 s. The dissociation condition was 10 μL/min×400 s and the regeneration conditions were 30 μL/min×30 s at pH1.5 Gly. FIG. 10 and FIG. 11 show the kinetic curves of bsAb-2-5 and bsAb-2-6 constructs bind to human and cynomolgus CD3 recombinant proteins, respectively. FIG. 12 show the kinetic curves of bsAb2-5 and bsAb2-6 constructs bind to HBsAg.

As shown in Table 3, FIG. 10 and FIG. 12, the exemplary HBsAg/CD3 bispecific antibodies of BsAb2-6-001, BsAb2-6-006, BsAb2-6-011, BsAb2-6-016, BsAb2-6-017 and BsAb2-5-001 had comparable binding affinity to HBsAg at the KD of 10\hat{ }⁻¹¹ M but with differential binding affinities to human CD38, ranging from 10\hat{ }^{8˜-10} M. All of these constructs show good cross-reactivity to cynomolgus CD38 (seen in FIG. 11), indicating which could be used as a non-human primate model for preclinical safety toxicity studies of anti-HBsAg/anti-CD3 bispecific antibodies.\

TABLE 3
Binding affinity of anti-HBsAg/anti-CD3 bispecific antibodies
to human CD3ε, cynomolgus CD3ε and HBsAg.

| | Human CD3ε | | | | Cynomolgus CD3ε | |
|---|---|---|---|---|---|---|
| ID | (RU²) | Kₒₙ ( ) | K_{off} | K_{D}(M) | (RU²) | Kₒₙ |
| SCG2-6-001 | 6.45E−01 | 4.44E+05 | 2.41E−04 | 7.68E−09 | 9.31E−01 | 5.76E+04 |
| SCG2-6-006 | 2.85E−01 | 6.44E+05 | 2.00E−04 | 3.11E−09 | 1.35E−01 | 1.68E+06 |
| SCG2-6-011 | 2.93E−01 | 3.33E+04 | 9.72E−03 | 2.92E−08 | 5.03E−01 | 4.37E+04 |
| SCG2-6-016 | 1.32E+00 | 2.34E+04 | 8.25E−03 | 3.53E−08 | 8.21E−02 | 9.73E+04 |
| SCG2-6-017 | 4.36E+02 | 3.00E+05 | 2.56E−04 | 8.51E−10 | 2.42E−01 | 2.78E+05 |
| SCG2-6-021 | 1.44E−01 | 1.35E+05 | 1.42E−03 | 10.5E−08 | 5.12E−01 | 2.76E+04 |
| SCG2-6-026 | 1.25E−01 | 8.48E+04 | 1.36E−03 | 1.60E−08 | 7.11E−01 | 3.86E+04 |
| SCG2-5-011 | 6.85E−01 | 9.30E+04 | 1.15E−03 | 1.24E−08 | 8.82E−01 | 3.72E+04 |

| | |
|---|---|
| Cynomolgus CD3ε | HBsAg |

| ID | K_{off} | K_{D}(M) | (RU²) | Kₒₙ | K_{off} | K_{D}(M) |
|---|---|---|---|---|---|---|
| SCG2-6-001 | 2.78E−03 | 4.83E−08 | 1.38E−01 | 4.63E+03 | 2.27E−07 | 4.91E−11 |
| SCG2-6-006 | 1.55E−02 | 9.23E−09 | 1.36E−01 | 2.24E+03 | 2.60E−09 | 1.16E−12 |
| SCG2-6-011 | 1.60E−03 | 3.67E−08 | 1.10E−01 | 5.01E+03 | 1.88E−06 | 3.75E−10 |
| SCG2-6-016 | 3.51E−03 | 3.60E−08 | 2.80E−01 | 6.08E+03 | 3.29E−07 | 5.40E−11 |
| SCG2-6-017 | 2.05E−04 | 7.37E−10 | 6.11E−02 | 1.30E+03 | 4.17E−08 | 3.21E−11 |
| SCG2-6-021 | 4.09E−03 | 1.48E−07 | 2.63E−01 | 4.49E+03 | 1.56E−06 | 3.48E−10 |
| SCG2-6-026 | 3.76E−03 | 9.74E−08 | 1.48E−01 | 2.63E+03 | 1.64E−06 | 6.23E−10 |
| SCG2-5-011 | 1.70E−03 | 4.56E−08 | 2.67E+00 | 2.48E+04 | 2.31E−06 | 9.32E−11 |

### Example 8 Purity Determination by Size-Exclusion Chromatography of Anti-HBsAg/Anti-CD3 Bispecific Antibodies

Bispecific antibodies generated in Example 1 are analyzed for purity determination by size-exclusion chromatography. SEC-HPLC was performed using a HPLC system (1260 Infinity II model, Agilent Technologies) and a XBridge BEH200 Å SEC Column (3.5 μm, 7.8×300 mm; Waters). Mobile phase A consisted of 67 mM Na₂HPO₄, 33 mM NaH₂PO₄, 100 mM Na₂SO₄ at pH 7.0. The flow rate was set to 0.5 mL/min of 100% mobile phase A at a column temperature of 30° C. for 30 mins. 100 μg of protein was injected on the column and detection occurred at 280 nm, 10 Hz. Data processing was performed using CDS2 (Agilent).

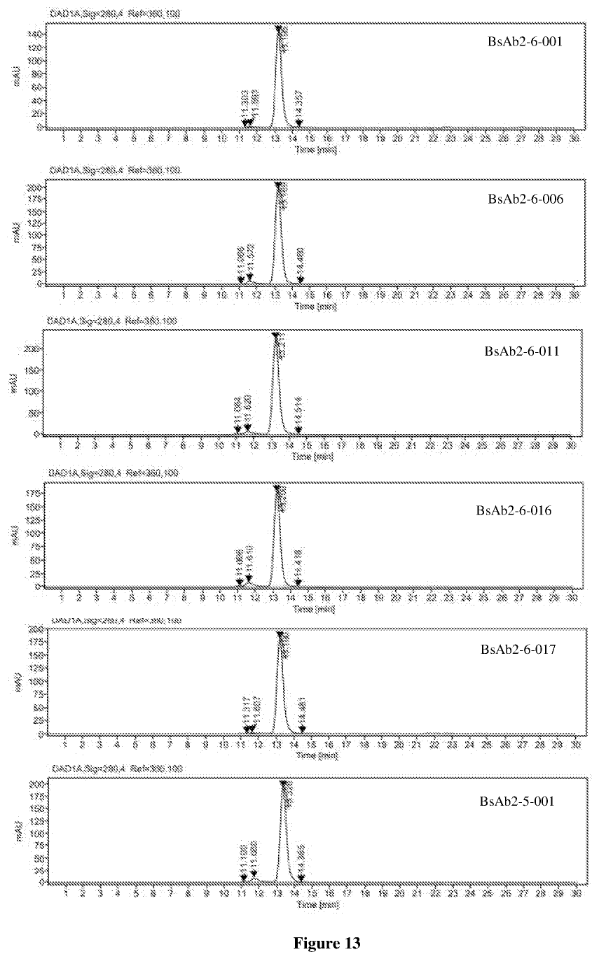

FIG. 13 illustrates that the high purity of the anti-HBsAg/anti-CD3 bispecific antibodies, suggesting that such molecules have good pharmacological exploitability.

### Example 9 In Vitro HBV Neutralizing Potency of Anti-HBsAg/Anti-CD3 Bispecific Antibodies

The anti-HBsAg/anti-CD3 bispecific antibodies generated in Example 1 are analyzed for their potency to neutralize HBV in vitro using Huh7-NTCP cells. Exactly, Huh7-NTCP cells were harvested into pre-warmed DMEM medium supplemented with 10% FBS and 2.5% DMSO at the density of 3.5E6 cells/mL. Then Huh7-NTCP were seeded into collagen-coated 96-well plates at 100 μL/well (1×10⁴ cells/well) and incubated overnight. The next day, HBV virus was diluted and incubated with 100, 10, 0 nM (only HBV virus as systematic control) bsAb2-6-011 and C8 mAb, then added to Huh7-NTCP cells, respectively. The second day, HBV virus was withdrawn and Huh7-NTCP cells were further incubated with fresh DMEM complete medium for 5˜10 days. Supernatant HBeAg concentration (readout as neutralization capacity) was determined according to the protocol of HBeAg ELISA kit.

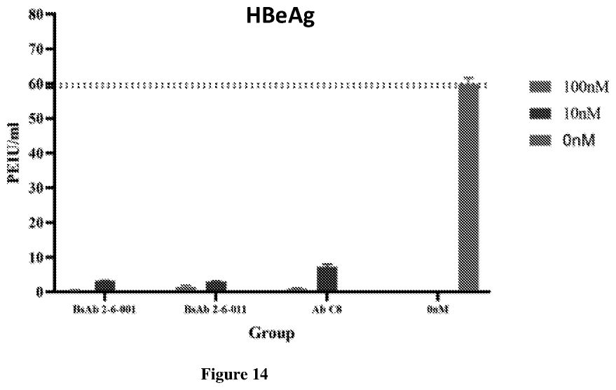

FIG. 14 show that both bsAb2-6-001 and bsAb2-6-011 exert a strong neutralizing activity at dosages of 10 nM and 100 nM, whereas the HBeAg value is as low as single digital PEIU/mL. This is consistent with the potency of parental C8 mAb. By comparison, the HBeAg value of HBV virus group is about 60 PEIU/MI, indicating a successfully infection for the assay system.

### Example 10 In Vivo Anti-Tumor Activities of Anti-HBsAg/Anti-CD3 Bispecific Antibodies on HBs⁺HepAD38 CDX Xenograft

The in vivo anti-tumor activities of anti-HBsAg/anti-CD3 bispecific antibodies generated in Example 1 are evaluated in immune deficient mice that are inoculated with HBs+HepAD38 cell line. In this model, HepAD38 cells (1E7 cells per mouse) were subcutaneously transplanted into the right flank of NPG mice (NOD.Prkdc^{acid}I12rg−/−) from VitalStar. After one week, mice were reconstituted with 1.5E7 human T cells purified from healthy donor. After two weeks, mice were grouped when tumors size reached 100˜200 mm³ and hCD45 ratio >5%. Treatment was initiated on day 0 by i.v. injection of bsAb2-6-011 at the dose of 0.3 mpk, 1 mpk twice per week, PBS treated group set as vehicle and anti-RSV IgG treated group set as isotype control. The tumor size and body weight was measured every 2-3 days. Tumor volume (mm³) was calculated from caliper measurement as: 0.5*length*width².

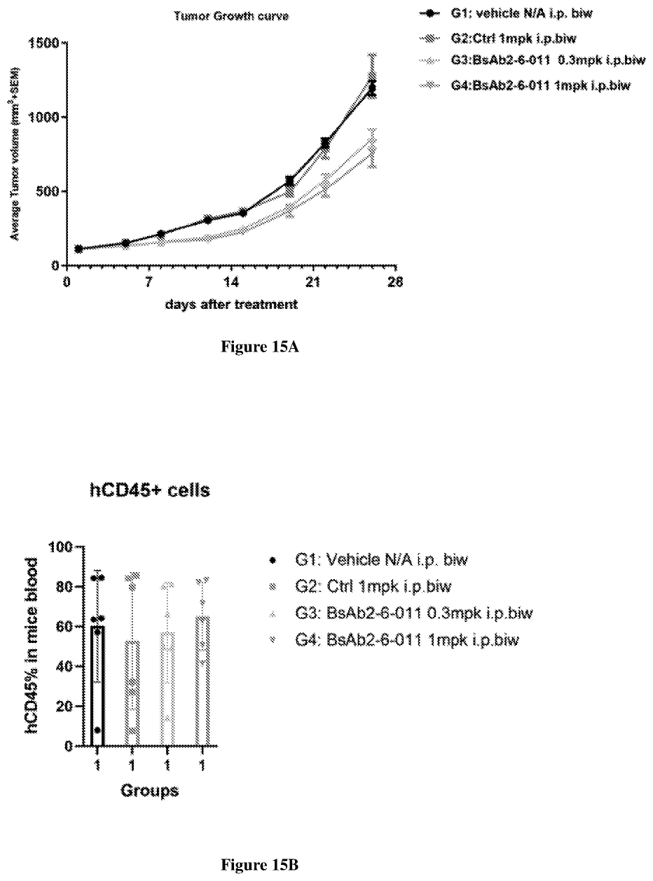

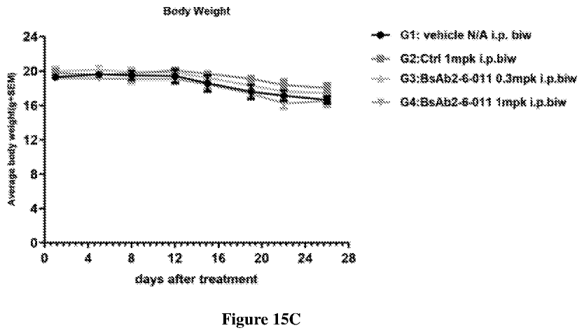

The results are shown in FIG. 15, bsAb2-6-011 efficiently reduced HepAD38 tumor size at the dosages of 0.3 mpk and 1 mpk., whereas the vehicle and isotype control group did not affect tumor growth (FIG. 15A). On day 26 after treatment, the average tumor size in mice that had been treated with bsAb2-6-011 was significantly smaller that in mice that had been treated with the PBS and isotype control.

In FIG. 15B, the percentages of human leukocytes in mice blood, as determined in the endpoint, are indicated. The percentage of circulating human leukocytes (hCD45+ cells) was comparable in all groups, whereas slightly enhancement was observed in mice treated with 1 mkp bsAb2-6-011, indicating that T cell activation after treated with the anti-HBsAg/anti-CD3 bispecific antibody. No major changes in mice bodyweight were observed in all treated groups (seen in FIG. 15C).

### Example 11 Pharmacokinetics Properties of Anti-HBsAg/Anti-CD3 Bispecific Antibodies

Pharmacokinetics in cynomolgus by one of the anti-HBsAg/anti-CD3 bispecific antibody is tested according to study described as below. In this study was performed at Pharmalegacy. Three male and three female cynomolgus monkeys were assigned to three groups receiving a single intravenous injection of bsAb2-6-011 at a dose of 0.1 mpk, 1 mpk and 5 mpk. The pharmacokinetic profile of bsAb2-6-011 was evaluated by determining plasma samples obtained pre-dose and at various time-points after dosing up to 7 days. The total concentration of bsAb2-6-011 was determined by Sandwich ELISA.

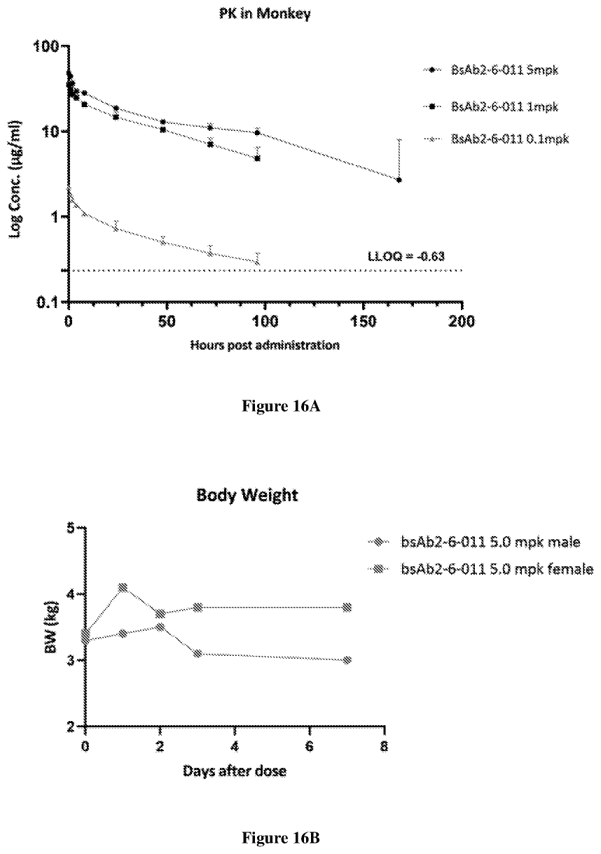

As shown in FIG. 16A, the pharmacokinetics profile of bsAb2-6-011 shows a fast initial distribution and clearance in cynomolgus monkeys, with T_{half}≈63 hrs. In addition, no significant bodyweight changes were observed in cynomolgus monkeys treated with 5 mpk bsAb2-6-011 (seen in FIG. 16B).

1. A bispecific antibody comprising:
(a) a first polypeptide chain comprising a heavy chain of the anti-CD3 antibody fused with a single chain fragment (scFv) binding to hepatitis B virus surface antigen (HBsAg), wherein the HBsAg comprises small surface antigen, hepatitis B virus medium surface antigen, or hepatitis B virus large surface antigen,
(b) a second polypeptide chain comprising a light chain of the anti-human CD3 antibody.

2. The bispecific antibody according to claim 1, wherein the HBsAg scFv is fused to the N-terminus or C-terminus of the heavy chain of the anti-CD3 antibody or fused to the heavy chain of the anti-CD3 antibody through a glycine-serine linker.

3. (canceled)

4. The bispecific antibody according to claim 2, wherein the glycine-serine linker is selected from (GGGGS)ₙ as shown in SEQ ID NO: 33, (GGGSG)ₙ as shown in SEQ ID NO: 34, (GSGGG)ₙ as shown in SEQ ID NO: 35, (GSGGGP)ₙ as shown in SEQ ID NO: 36, (GSEPS)ₙ as shown in SEQ ID NO: 37, (GGEGGGP)ₙ as shown in SEQ ID NO: 38, (GGEGGGSEGGGS)ₙ as shown in SEQ ID NO: 39, (GGGSGGGG)ₙ as shown in SEQ ID NO: 40 or a combination thereof, wherein the n=1, 2, 3, 4, 5, 6, 7, 8, 9 or 10.

5. The bispecific antibody according to claim 1, wherein the HBsAg scFv comprises the HCDR1, HCDR2, HCDR3, LCDR1, LCDR2, and LCDR3 as shown in SEQ ID NO: 1 to SEQ ID NO: 6, respectively, and/or
the CD3 antibody comprises the HCDR1, HCDR2, HCDR3, LCDR1, LCDR2, and LCDR3 as shown in SEQ ID NO: 11 to SEQ ID NO: 16, respectively.

6. The bispecific antibody according to claim 5, wherein the scFv comprises a VH binding domain and a VL binding domain; wherein the VH comprises the amino acid sequence that is at least 90% identical to SEQ ID NO: 7; the VL comprises the amino acid sequence that is at least 90% identical to SEQ ID NO: 8.

7. The bispecific antibody according to claim 6, wherein the VH domain and VL domain are linked by a “YOL” linker; preferably, the “YOL” linker comprises the amino acid sequence of SEQ ID NO: 9.

8. The bispecific antibody according to claim 7, wherein the scFv comprises the amino acid sequence that is at least 90% identical to SEQ ID NO: 10.

9. The bispecific antibody according to claim 1, wherein the anti-CD3 antibody comprises a VH binding domain and a VL binding domain; wherein the VH comprises the amino acid sequence that is at least 90% identical to SEQ ID NO: 19, SEQ ID NO: 20, SEQ ID NO: 21, SEQ ID NO: 22, SEQ ID NO: 23, or SEQ ID NO: 24; the VL comprises the amino acid sequence that is at least 90% identical to SEQ ID NO: 25, SEQ ID NO: 26, SEQ ID NO: 27, SEQ ID NO: 28, or SEQ ID NO: 29.

10. The bispecific antibody according to claim 1, wherein the bispecific antibody is an IgG1-kappa isotype.

11. The bispecific antibody according to claim 1, wherein the bispecific antibody further comprises alanine at position 234 and alanine at position at 235 of heavy chain, wherein residue numbering is according to the EU Index.

12. The bispecific antibody according to claim 1, wherein,
the first polypeptide chain comprising amino acid sequence of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO: 52; and/or
the second polypeptide chain comprising amino acid sequence of SEQ ID NO: 53, SEQ ID NO: 54, SEQ ID NO: 55, SEQ ID NO: 56, or SEQ ID NO: 57.

13. The bispecific antibody according to claim 1, which is encoded in a nucleic acid molecule.

14. The bispecific antibody according to claim 13, wherein the nucleic acid molecule is comprised in an expression vector.

15. The bispecific antibody according to claim 1, which is comprised in a host cell.

16. The bispecific antibody according to claim 1, which is comprised in a pharmaceutical composition comprising a pharmaceutically acceptable excipient, diluent or carrier.

17. A method of treating diseases caused by HBV infection, the method comprising administering to a patient the bispecific antibody according to claim 1.

18. (canceled)

19. A CD3 antibody or an antigen-binding fragment thereof, comprising:
VH binding domain and VL binding domain; wherein the VH comprises the amino acid sequence that is at least 90% identical to SEQ ID NO: 19, SEQ ID NO: 20, SEQ ID NO: 21, SEQ ID NO: 22, SEQ ID NO: 23, or SEQ ID NO: 24; and/or;
the VL comprises the amino acid sequence that is at least 90% identical to SEQ ID NO: 25, SEQ ID NO: 26, SEQ ID NO: 27, SEQ ID NO: 28, or SEQ ID NO: 29.

20. The CD3 antibody or an antigen-binding fragment thereof according to claim 19, wherein the amino acid sequence of the VH domain as shown in SEQ ID NO: 19, SEQ ID NO: 20, SEQ ID NO: 21, SEQ ID NO: 22, SEQ ID NO: 23, or SEQ ID NO: 24; and
the amino acid sequence of the VL domain as shown in SEQ ID NO: 25.

21. The CD3 antibody or an antigen-binding fragment thereof according to claim 19, which is comprised in a bispecific antigen-binding molecule or is encoded in a nucleic acid molecule; or which is comprised in a bispecific antigen-binding molecule, and wherein the bispecific antigen-binding molecule is encoded in a nucleic acid molecule.

22. The CD3 antibody or an antigen-binding fragment thereof according to claim 20, which is encoded in a nucleic acid molecule.

23. (canceled)
